(19) United States
(12) Patent Application Publication
(10) Pub. No.: US 2012/0087940 A1
(43) Pub. Date: Apr. 12, 2012

(54) VECTOR FOR TREATING ALZHEIMER'S DISEASE

(75) Inventors: Makoto Inoue, Ibaraki (JP); Koichi Saeki, Ibaraki (JP); Jun You, Ibaraki (JP); Toshiaki Tabata, Ibaraki (JP); Hitoshi Iwasaki, Ibaraki (JP); Tsugumine Shu, Ibaraki (JP); Mamoru Hasegawa, Ibaraki (JP)

(73) Assignee: DNAVEC Corporation, Tsukuba-shi (JP)

(21) Appl. No.: 13/126,293

(22) PCT Filed: Oct. 30, 2009
(86) PCT No.: PCT/JP2009/068678

§ 371 Date: Dec. 16, 2011

(30) Foreign Application Priority Data
Oct. 31, 2008 (JP) 2008-282150

(51) Int. Cl. A61K 31/7105; A61P 25/28; C12N 15/86; A61K 39/00

(52) U.S. Cl. 424/193.1; 514/44 R; 424/184.1; 435/320.1

(57) ABSTRACT

The present invention provides methods for efficiently inducing anti-Aβ antibody and methods for preventing and treating Alzheimer's disease. The present inventors successfully induced anti-Aβ antibody in a highly efficient manner by administering an RNA viral vector that expresses a fusion protein between an AB5 toxin B subunit and an Aβ antigen peptide. Administration of the vector resulted in a significant increase of anti-Aβ antibody in plasma, and decrease in the Aβ level in brain tissues and decrease in the anti-Aβ antibody-positive area. The present invention enables more efficient vaccine gene therapy for preventing and treating Alzheimer's disease.

### TECHNICAL FIELD

The present invention relates to novel gene transfer vectors and vaccines for active immunization comprising the vectors and such, which are aimed to efficiently induce anti-Aβ antibodies, and prevent or treat Alzheimer's disease.

### BACKGROUND ART

The number of patients with a neurodegenerative disease has steadily increased as we transition to the so-called aging society. For example, there are one million and 4.5 million Alzheimer's disease patients in Japan and the United States, respectively. The number of Alzheimer's disease patients worldwide is estimated to be 15 millions or more, and is predicted to double or more in the next two decades. Some therapeutic agents are currently available. However, there remains a need for development of therapeutic methods that are applicable in advanced stages of the disease, therapeutic methods that are highly effective to block the progression in earlier stages, methods for preventing the onset itself, etc. Thus, the social demands for such novel therapeutic methods are extremely high.

The leading hypothesis for pathogenesis of Alzheimer's disease is the “amyloid cascade hypothesis” which assumes that the cause is senile plaque formation due to aggregation and deposition of amyloid β (Aβ). A vaccine therapy targeted to Aβ based on this hypothesis has drawn attention as a novel therapeutic method for treating Alzheimer's disease, and induction of the humoral immunity (anti-Aβ antibody) is considered important for its effectiveness. Indeed, it has been reported that the brain amyloid deposition was reduced in model mice when aggregated Aβ peptide was administered along with an adjuvant (Schenk D, et al., Nature 400:173-177, 1999).

Based on the findings described above, Elan and Wyeth Corp. conducted clinical trials of synthetic Aβ peptide (AN-1792; Aβ42), which was administered along with an adjuvant (QS21). It was reported that the serum anti-Aβ antibody level was elevated in about 20% of the subjects (Hock C. et al., Nat. Med. 8:1270-1275, 2002); the higher-order brain functions were ameliorated (Hock C. et al., Neuron 38:547-554, 2003); and the effectiveness was confirmed by long-term observation (Gilman S. et al., Neurology 64:1553-1562, 2005). On the other hand, the vaccine therapy using synthetic Aβ peptide in combination with an adjuvant has been reported to occasionally cause meningoencephalitis (Orgogozo J M, et al., Neurology 61:46-54, 2003). A pathological tissue analysis of brains with meningoencephalitis revealed that growth of astrocytes and disappearance of degenerated axons were observed after disappearance of neocortical senile plaques in some cases. A speculative cause of the onset of meningoencephalitis is that in some patients, cellular immunity was induced by adjuvant which is required in vaccine therapy, and as a result Th1-type CD4-positive T cells reactive to Aβ or APP infiltrated into the brain and caused experimental allergic encephalomyelitis-like meningoencephalitis. The vaccine therapy itself is recognized as being effective. Thus, there is a need to develop safer vaccination techniques that do not cause meningoencephalitis.

A method that uses only the N terminal portion of Aβ peptide which is predicted not to contain any T cell epitope was devised and assessed as a means to suppress meningoencephalitis (side effect). An immunization method that uses the Aβ1-7 peptide in combination with a Th2-type adjuvant (CRM197; non-toxic diphtheria toxin mutant) has been developed (Vaccine ACC-001; Wyeth) and assessed for its effectiveness. Furthermore, the Aβ1-15 peptide has also been assessed and was reported to be a weaker immunogen than Aβ42 (Leverone J F et al., Vaccine 21:2197-206, 2003). However, the titer of anti-Aβ antibody was demonstrated to be increased by using a dendrimer of Aβ1-15 (Seabrook T J et al., J Neuroinflammation 3:14, 2006; Seabrook T J et al., Neurobiol Aging 28:813-23, 2007). In addition, two-tandem-type Aβ1-15 was also revealed to slightly increase the titer of anti-Aβ antibody and to be effective in model mice (Maier M et al., J Neurosci. 26:4717-4728, 2006).

As described above, using only the N terminal portion of Aβ peptide is thought to be an effective means for increasing safety. However, the N terminal portion functions only weakly as an immunogen. Thus, there is a need to employ a combination of improvements on the structure, adjuvant, administration method, and the like.

Clinical trials (bapineuzumab, also referred to as AAB-001; Elan and Wyeth Corp; RN-1219, Rinat/Pfizer Corp.; LY-2062430, Eli Lilly Corp.) have also been conducted to assess a passive immunization method based on direct administration of an antibody against Aβ (Bard F, Cannon C, Barbour R et al., Nat. Med. 6:916-919, 2000). In theory, this method does not induce T cell reaction. Therefore, unlike AN1792, the method is expected not to cause meningoencephalitis. However, passive immunization imposes a risk of generating anti-idiotype antibodies from long term administration of a large amount of antibody and a risk of hemorrhagic tendency due to amyloid deposition in blood vessels.

Meanwhile, gene vaccination has also been assessed, and reported examples of gene vaccination include those using plasmids (Qu B, Boyer P J, Johnston S A et al., J Neurol Sci. 244:151-158, 2006; Okura Y, Miyakoshi A, Kohyama K et al., Proc. Natl. Acad. Sci. USA, 103:9619-9624, 2006), adenovirus vectors (Kim H D, Cao Y, Kong F K et al., Vaccine 23:2977-2986, 2005; Kim H D, Tahara K, Maxwell J A et al., J Gene Med. 9:88-98, 2007), and adeno-associated virus vectors (Zhang J, Wu X, Qin C et al., Neurobiol Dis. 14:365-379, 2003; Hara H, Monsonego A, Yuasa K et al., J Alzheimers Dis. 6:483-488, 2004). However, gene vaccination is still insufficient in terms of efficacy.

As described above, the vaccine therapy itself has been recognized as being effective; however, there is no report published on the establishment of safer and more effective vaccination techniques. Thus, there are currently strong demands for the development of such techniques.

### PRIOR-ART DOCUMENTS

### Non-Patent Documents

Non-patent Document 1: Schenk D. et al., Nature 400:173-177, 1999
Non-patent Document 2: Hock C. et al., Nat. Med. 8:1270-1275, 2002
Non-patent Document 3: Hock C. et al., Neuron 38:547-554, 2003
Non-patent Document 4: Gilman S. et al., Neurology 64:1553-1562, 2005
Non-patent Document 5: Orgogozo J M. et al., Neurology 61:46-54, 2003
Non-patent Document 6: Leverone J F. et al., Vaccine 21:2197-206, 2003
Non-patent Document 7: Seabrook T J. et al., J Neuroinflammation 3:14, 2006
Non-patent Document 8: Seabrook T J. et al., Neurobiol Aging 28:813-23, 2007
Non-patent Document 9: Maier M. et al., J Neurosci. 26:4717-4728, 2006
Non-patent Document 10: Bard F. et al., Nat. Med. 6:916-919, 2000
Non-patent Document 11: Qu B. et al., J Neurol Sci. 244:151-158, 2006
Non-patent Document 12: Okura Y. et al., Proc. Natl. Acad. Sci. USA, 103:9619-9624, 2006
Non-patent Document 13: Kim H D. et al., Vaccine 23:2977-2986, 2005
Non-patent Document 14: Kim H D. et al., J Gene Med. 9:88-98, 2007
Non-patent Document 15: Zhang J. et al., Neurobiol Dis. 14:365-379, 2003
Non-patent Document 16: Hara H. et al., J Alzheimers Dis. 6:483-488, 2004

### DISCLOSURE OF THE INVENTION

### Problems to be Solved by the Invention

The present invention was achieved in view of the circumstances described above. An objective of the present invention is to provide methods for efficiently inducing an anti-Aβ antibody, and safer and more effective immunotherapy methods for treating or preventing Alzheimer's disease.

### Means for Solving the Problems

To achieve the above-described objective, the present inventors conducted dedicated studies to develop vaccine therapy using RNA viral vectors for expressing Aβ. During the course, the present inventors discovered that the antibody against Aβ was induced at a significantly high titer by vaccine therapy using an RNA viral vector in combination with a nucleic acid that expresses a fusion protein between an AB5 toxin B subunit and an amyloid β peptide. In particular, an RNA viral vector that expresses a fusion protein containing the amyloid β peptide Aβ1-15 produced a significantly efficacious therapeutic effect as compared to conventional therapeutic methods. As compared to the amyloid β expression vector previously reported by the present inventors (WO 2006/112553), the viral vector achieved a markedly increased expression level of Aβ and enabled secure expression of anti-Aβ antibody which is an indicator of effectiveness. Furthermore, meningoencephalitis, which was a problem when immunization was performed using the conventional combination of Aβ peptide and adjuvant, was not observed with the developed therapeutic method. This suggests that the therapeutic method can secure safety.

Specifically, the present invention relates to RNA viral vectors encoding a fusion protein between an AB5 toxin B subunit and an amyloid β antigen peptide, induction of anti-Aβ antibodies (humoral immunity) using the vectors, prevention and treatment of Alzheimer's disease using the vectors, etc. More specifically, the present invention relates to:

[1] an RNA viral vector encoding a fusion protein between an AB5 toxin B subunit and an amyloid β antigen peptide;
[2] the vector of [1], wherein the AB5 toxin B subunit is a cholera toxin B (CTB);
[3] the vector of [1] or [2], wherein the amyloid β antigen peptide comprises one or more copies of Aβ1-15 or a fragment thereof;
[4] the vector of [3], wherein the amyloid β antigen peptide has a structure in which one to eight copies of Aβ1-15 or fragments thereof are linked together;
[5] the vector of [4], wherein the amyloid β antigen peptide has a structure in which four to eight copies of Aβ1-15 are linked together;
[6] the vector of any one of [1] to [5], wherein the RNA viral vector is a minus-strand RNA viral vector;
[7] the vector of [6], wherein the minus-strand RNA viral vector is a paramyxovirus vector;
[8] the vector of [7], wherein the paramyxovirus vector is a Sendai virus vector;
[9] a composition comprising the vector of any one of [1] to [8] and a pharmaceutically acceptable carrier;
[10] the composition of [9] for inducing an anti-Aβ antibody;
[11] the composition of [9] or [10] for preventing or treating Alzheimer's disease;
[12] a pharmaceutical agent for preventing or treating Alzheimer's disease, which comprises the vector of any one of [1] to [8];
[13] use of the vector of any one of [1] to [8] in producing a composition for inducing an anti-Aβ antibody;
[14] use of the vector of any one of [1] to [8] in producing a pharmaceutical composition for preventing or treating Alzheimer's disease;
[15] the use of [13] or [14], wherein booster immunization is carried out using a protein comprising an amyloid β antigen or a vector encoding the protein;
[16] the use of [15], wherein the protein comprising an amyloid β antigen is a fusion protein between an AB5 toxin B subunit and an amyloid β antigen peptide;
[17] a method for inducing an anti-Aβ antibody, which comprises the step of administering the vector of any one of [1] to [8] or a composition comprising the vector and a pharmaceutically acceptable carrier;
[18] a method for preventing or treating Alzheimer's disease, which comprises the step of administering the vector of any one of [1] to [8] or a composition comprising the vector and a pharmaceutically acceptable carrier;
[19] the method of [17] or [18], which additionally comprises the step of administering a protein comprising an amyloid β antigen or a vector encoding the protein for booster immunization;
[20] the method of [19], wherein the protein comprising an amyloid β antigen is a fusion protein between an AB5 toxin B subunit and an amyloid β antigen peptide;
[21] a method for increasing the titer of an antibody against an antigen protein, which comprises the step of administering an RNA viral vector encoding the antigen two or more times;
[22] a composition for increasing the titer of an antibody against an antigen protein by a method that comprises the step of administering two or more times a vector that comprises an RNA viral vector encoding the antigen and a pharmaceutically acceptable carrier;
[23] use of an RNA viral vector encoding an antigen protein in producing an agent for increasing the titer of an antibody against the antigen by a method that comprises the step of administering the vector two or more times; and
[24] a virus-like particle comprising the vector of any one of [1] to [8].

It is intended that in each of the claims described above, inventions comprising any combination of two or more inventions described in each claim that recites the same claim are also included in the antecedent claim recited. Furthermore, it is intended that any inventive elements and technical elements described herein, and any combinations thereof are also included in the present invention. In addition, it is intended that any inventions excluding any elements described herein or any combinations thereof are also included in the present invention. Herein, for example, when a specific embodiment is stated as “preferable”, the specification discloses not only the embodiment itself, but also inventions that exclude the embodiment from the disclosed antecedent inventions comprising the embodiment.

### Effects of the Invention

Vaccine therapy for Alzheimer's disease using the vectors of the present invention does not only help patients with the Alzheimer-type dementia, for which there has been no effective therapeutic method. Many social contributions such as reduction of medical costs and significant improvement in the quality of life and problems of nursing care in elderly persons are also expected. Meanwhile, dedicated studies have been recently conducted to develop technologies for early diagnosis of Alzheimer's disease using positron CT (PET) or magnetic resonance imaging (MRI). Some of the technologies are already under clinical investigation. The burdens on patients and their families as well as social burdens can be expected to be considerably reduced when radical therapy is provided at an early stage of onset by using the highly effective vaccine therapy of the present invention in combination with early diagnosis.

### BRIEF DESCRIPTION OF THE DRAWINGS

FIG. 1 is a schematic diagram showing the structure of the Aβ42 NotI fragment.

FIG. 2 is a schematic diagram showing construction of the Aβ42 NotI fragment.

FIG. 3 is a schematic diagram showing the structure of the CTB-Aβ42 NotI fragment.

FIG. 4 is a diagram showing the expression quantity of Aβ42, IL-4-Aβ42, PEDI-Aβ42, and CTB-Aβ42 in cell lysates and culture supernatants of BHK21 cells.

FIG. 5 is a schematic diagram showing construction of the CTB-Aβ15 NotI fragment.

FIG. 6 is a schematic diagram showing construction of the CTB-Aβ15×2, CTB-Aβ15×4, and CTB-Aβ15×8 NotI fragments.

FIG. 7 is a Western blot photograph showing the amount of Aβ antigen in cell lysates and culture supernatants of BHK cells infected with an SeV vector carrying the CTB-Aβ42, CTB-Aβ15, CTB-Aβ15×2, CTB-Aβ15×4, or CTB-Aβ15×8 gene.

FIG. 8 is a diagram showing the GM1-binding activity in cell lysates and culture supernatants of BHK cells infected with an SeV vector carrying the CTB-Aβ42, CTB-Aβ15, CTB-Aβ15×2, CTB-Aβ15×4, or CTB-Aβ15×8 gene.

FIG. 9 is a diagram showing the titer of anti-Aβ antibody in C57BL/6N mice intramuscularly administered with an SeV vector carrying the CTB-Aβ42, CTB-Aβ15×8, or GFP gene.

FIG. 10 is a diagram showing the titer of anti-Aβ antibody in C57BL/6N mice intramuscularly, intradermally, or intranasally administered with an SeV vector carrying the CTB-Aβ15×8 gene.

FIG. 11 is a diagram showing the titer of anti-Aβ antibody in C57BL/6N mice intranasally administered with an SeV vector carrying the CTB-Aβ15, CTB-Aβ15×2, CTB-Aβ15×4, or CTB-Aβ15×8 gene.

FIG. 12 is a graph showing the titer of anti-Aβ antibody in C57BL/6N mice intramuscularly administered with an SeV vector carrying the CTB-Aβ42 gene and then subjected to booster immunization with the CTB-Aβ42 protein purified from E. coli.

FIG. 13 is a graph showing the titer of anti-Aβ antibody in C57BL/6N mice intramuscularly administered with an SeV vector carrying the CTB-Aβ15×8 gene and then subjected to booster immunization with the same SeV vector.

FIG. 14 shows in graphs the titer of anti-Aβ antibody in C57BL/6N mice intramuscularly administered with an SeV vector carrying the CTB-Aβ42 or CTB-Aβ15×8 gene and then subjected to booster immunization with the same SeV vector. Panel B shows only results obtained by using the SeV vector carrying the CTB-Aβ42 gene shown in panel A.

FIG. 15 shows in graphs the titer of anti-Aβ antibody in C57BL/6N mice intranasally administered with an SeV vector carrying the CTB-Aβ15×8 gene and then subjected to booster immunization with the same SeV vector.

FIG. 16 shows in a graph the titer of anti-Aβ antibody in Tg2576 mice intramuscularly administered with an SeV vector carrying the CTB-Aβ15×8, CTB-Aβ42, or GFP gene and then subjected to booster immunization with the CTB-Aβ42 protein.

FIG. 17 shows in diagrams the intracerebral quantity of Aβ in Tg2576 mice intramuscularly administered with an SeV vector carrying the CTB-Aβ15×8, CTB-Aβ42, or GFP gene and then subjected to booster immunization with the CTB-Aβ42 protein.

FIG. 18 shows in photographs the distribution of areas of positive 6E10 immunostaining in histopathological brain samples. In the control SeV18+GFP/ΔF group (A), many dispersed Aβ deposits (brown) positive for 6E10 staining are seen in the olfactory bulb, hippocampus, and cerebral neocortex. In contrast, the number of Aβ deposits is clearly smaller in the SeV18+CTB-Aβ15×8/ΔF administration group (B).

FIG. 19 is a graph of image analysis of samples immunostained with 6E10. In the SeV18+CTB-Aβ15×8/ΔF administration group (right), there is an apparent reducing trend of percent area in various brain regions as compared to the control SeV18+GFP/ΔF group (left). In particular, there is a great difference in the hippocampus.

FIG. 20 shows in a diagram induction of anti-Aβ antibody in normal mice by using proteins. In group A (six heads), SeV18+GFP/ΔF was intramuscularly administered at 5×10⁷ CIU/200 μl/head, and eight weeks later the same vector was administered in the same manner. In group B (six heads), SeV18+CTB-Aβ15×4KK/ΔF was intramuscularly administered at 5×10⁷ CIU/200 μl/head, and eight weeks later the same vector was administered in the same manner. In group C (six heads), SeV18+CTB-Aβ15×4KK/ΔF was intramuscularly administered at 5×10⁷ CIU/200 μl/head, and then the CTB-Aβ15×4KK protein was subcutaneously administered at 100 μg/100 μl/head once every two weeks for a total of five times. In group D (six heads), the CTB-Aβ15×4KK protein was subcutaneously administered at 100 μg/100 μl/head, and then the CTB-Aβ15×4KK protein was subcutaneously administered at 100 μg/100 μl/head once every two weeks for a total of five times.

FIG. 21 shows in a diagram induction of anti-Aβ antibody in PDGF-hAPPV717I mice by using proteins. Group A is an untreated group. In group B, SeV18+CTB-Aβ15×4KK/ΔF was intranasally administered at 5×10⁷ CIU/10 μl/head, and eight weeks later the same vector was administered in the same manner. In group C, SeV18+CTB-Aβ15×4KK/ΔF was intranasally administered at 5×10⁷ CIU/10 μl/head, and then the CTB-Aβ15×4KK protein (produced in E. coli) was intranasally administered at 100 μg/15×2 μl/head once every two weeks for a total of seven times. In group D, CTB-Aβ15×4KK protein was intranasally administered at 100 μg/15×2 μl/head, and then the CTB-Aβ15×4KK protein was intranasally administered at 100 μg/15×2 μl/head once every two weeks for a total of seven times.

FIG. 22 shows in a diagram induction of anti-Aβ antibody in Tg2576 mice by the combined use of SeV and proteins. Group A is an untreated group. In group B, SeV18+GFP/ΔF was intranasally administered at 5×10⁷ CIU/200 μl/head, and 12 weeks later the same vector was administered in the same manner. In group C, SeV18+CTB-Aβ15×4KK/ΔF was intranasally administered at 5×10⁷ CIU/10 μl/head, and then the CTB-Aβ15×4KK protein was subcutaneously administered at 100 μg/100 μl/head once every week for a total of four times, and then once every two weeks for a total of five times.

FIG. 23 shows in diagrams decrease in the number of intracerebral senile plaques in Tg2576 mice by the combined use of SeV and proteins. Group A is an untreated group. In group B, SeV18+GFP/ΔF was intranasally administered at 5×10⁷ CIU/200 μl/head, and 12 weeks later the same vector was administered in the same manner. In group C, SeV18+CTB-Aβ15×4KK/ΔF was intranasally administered at 5×10⁷ CIU/10 μl/head, and then the CTB-Aβ15×4KK protein was subcutaneously administered at 100 μg/100 μl/head once every week for a total of four times, and then once every two weeks for a total of five times.

FIG. 24 shows in diagrams reduction in the intracerebral quantity of Aβ (insoluble Aβ42) in Tg2576 mice by the combined use of SeV and proteins. Group A is an untreated group. In group B, SeV18+GFP/ΔF was intranasally administered at 5×10⁷ CIU/200 μl/head, and 12 weeks later the same vector was administered in the same manner. In group C, SeV18+CTB-Aβ15×4KK/ΔF was intranasally administered at 5×10⁷ CIU/10 μl/head, and then the CTB-Aβ15×4KK protein was subcutaneously administered at 100 μg/100 μl/head once every week for a total of four times, and then once every two weeks for a total of five times.

FIG. 25 shows in a diagram induction of anti-Aβ antibody in PDGF-hAPPV717I mice by various vectors. In group A, AAV-GFP was intramuscularly administered at 5×10¹⁰ particles/200 μl/head, and eight weeks later the same vector was administered in the same manner. In group B, AAV-CTBAβ42 was intramuscularly administered at 5×10¹⁰ particles/200 μl/head, and eight weeks later the same vector was administered in the same manner. In group C, SeV18+GFP/ΔF was intramuscularly administered at 5×10⁷ CIU/200 μl/head, and eight weeks later the same vector was administered in the same manner. In group D, SeV18+(CTB-Aβ42)/ΔF was intramuscularly administered at 5×10⁷ CIU/200 μl/head, and eight weeks later the same vector was administered in the same manner. In group E, SeV18+CTB-Aβ15×4KK/ΔF was intramuscularly administered at 5×10⁷ CIU/200 μl/head, and eight weeks later the same vector was administered in the same manner. In group F, SeV18+CTB-Aβ15×4KK/ΔF was intranasally administered at 5×10⁷ CIU/10 μl/head, and eight weeks later the same vector was administered in the same manner.

FIG. 26 shows in a diagram induction of anti-Aβ antibody in normal mice by non-infectious particles (VLP). In group A (six heads), SeV18+GFP/ΔF was intramuscularly administered at 5×10⁷ CIU/200 μl/head, and then the same vector was administered in the same manner once every week for a total of four times, and then once after two weeks. In group B (six heads), SeV18+(NP-Aβ15×8)/ΔF-VLP, which is a non-infectious particle, was intramuscularly administered at 150 μg/200 μl/head, and then the same vector was administered in the same manner once every week for a total of four times, and then once after two weeks.

### MODE FOR CARRYING OUT THE INVENTION

The present invention relates to RNA viral vectors encoding a fusion protein between an AB5 toxin B subunit (AB5B) and an antigen peptide derived from amyloid β (Aβ), anti-Aβ antibody-inducers comprising the vectors, compositions for preventing or treating Alzheimer's disease, methods for inducing anti-Aβ antibody using the vectors, and methods for preventing or treating Alzheimer's disease, etc. The present inventors revealed that the vaccination effect against Alzheimer's disease could be significantly enhanced by using an RNA viral vector in combination with the nucleic acid encoding a fusion protein of AB5B and Aβ. Specifically, the production of antibodies against Aβ can be drastically increased by using an RNA viral vector encoding a fusion protein of AB5B and Aβ peptide. Thus, the present invention enables novel effective therapy for preventing and/or treating Alzheimer's disease.

Herein, the viral vector refers to a vector (vehicle) that has a genomic nucleic acid derived from a virus and is used to express a gene inserted into the genomic nucleic acid. The viral vector of the present invention also includes complexes such as infectious virions, viral core, complex of viral genome and proteins, and non-infectious particles (non-infectious virions or virus-like particles), which have an ability to express inserted genes when they are introduced into cells. For example, in the case of an RNA virus, the ribonucleoprotein (the viral core) consisting of the viral genome and viral proteins that bind to the genome is capable of expressing an inserted gene in cells when it is introduced into the cells (WO 00/70055). Such ribonucleoproteins (RNPs) are also included in the viral vectors of the present invention. The vectors can be appropriately introduced into cells by using transfection reagents or the like. The introduced RNPs express genes inserted into the genomic RNA by the same mechanism as that of the original virus.

Herein, the RNA virus refers to a virus that has RNA genome and does not have any DNA phase in its life cycle. The RNA virus of the present invention has no reverse transcriptase (specifically, the RNA virus does not include retroviruses). Specifically, the viral propagation is achieved by replication of viral genome using RNA-dependent RNA polymerase and is not mediated by DNA. The RNA virus includes single-stranded RNA viruses (including both plus- and minus-strand RNA viruses) and double-stranded RNA viruses. The RNA viruses include viruses with envelopes (enveloped viruses) and viruses without envelope (non-enveloped viruses). Vectors derived from an enveloped virus are preferred. Specifically, the RNA viruses of the present invention include viruses belonging to the following viral families:

Arenaviridae, including Lassa virus;
Orthomyxoviridae, including influenza viruses (including Influenza virus A, B, and C, and Thogoto-like viruses);
Coronaviridae, including SARS virus;
Togaviridae, including rubella virus;
Paramyxoviridae, including mumps virus, measles virus, Sendai virus, and RS virus;
Picornaviridae, including poliovirus, Coxsackievirus, and echovirus;
Filoviridae, including Marburg virus and Ebola virus;
Flaviviridae, including yellow fever virus, dengue fever virus, hepatitis C virus, and hepatitis G virus;
Bunyaviridae (including the genera Bunyavirus, Hantavirus, Nairovirus, Phlebovirus, etc.);
Rhabdoviridae, including rabies virus; and

### Reoviridae.

Preferred RNA viral vectors of the present invention include, for example, minus-strand RNA viral vectors. A minus-strand RNA viral vector refers to a viral vector derived from a virus containing a minus-strand (an antisense strand that encodes viral proteins) RNA as the genome. The minus-strand RNA is also referred to as negative strand RNA. In particular, the minus-strand RNA viruses of the present invention include single-stranded minus-strand RNA viruses (also referred to as non-segmented minus-strand RNA viruses). The “single-stranded negative strand RNA virus” refers to viruses having a single-stranded negative strand (i.e., a minus strand) RNA as the genome. Such viruses include viruses belonging to Paramyxoviridae (including the genera Paramyxovirus, Morbillivirus, Rubulavirus, and Pneumovirus), Rhabdoviridae (including the genera Vesiculovirus, Lyssavirus, and Ephemerovirus), Filoviridae, and taxonomically belong to Mononegavirales (Virus, Vol. 57(1):29-36, 2007; Annu. Rev. Genet. 32:123-162, 1998; Fields Virology Fourth edition, Philadelphia, Lippincott-Raven, 1305-1340, 2001; Microbiol. Immunol. 43:613-624, 1999; Field Virology, Third edition pp. 1205-1241, 1996).

In particular, the minus-strand RNA viral vectors of the present invention include paramyxovirus vectors. The paramyxovirus vectors are viral vectors derived from a virus belonging to Paramyxoviridae. Such viruses include, for example, Sendai virus belonging to Paramyxoviridae. The viruses also include, for example, Newcastle disease virus, mumps virus, measles virus, respiratory syncytial (RS) virus, rinderpest virus, distemper virus, simian parainfluenza virus (SV5), human parainfluenza viruses 1, 2, and 3; influenza virus belonging to Orthomyxoviridae; and vesicular stomatitis virus and rabies virus belonging to Rhabdoviridae.

The viruses that can be used in the present invention also include, for example, Sendai virus (SeV), human parainfluenza virus-1 (HPIV-1), human parainfluenza virus-3 (HPIV-3), phocine distemper virus (PDV), canine distemper virus (CDV), dolphin molbillivirus (DMV), peste-des-petits-ruminants virus (PDPR), measles virus (MV), rinderpest virus (RPV), Hendra virus (Hendra), Nipah virus (Nipah), human parainfluenza virus-2 (HPIV-2), simian parainfluenza virus 5 (SV5), human parainfluenza virus-4a (HPIV-4a), human parainfluenza virus-4b (HPIV-4b), mumps virus (Mumps), and Newcastle disease virus (NDV). More preferably, the viruses of the present invention include viruses selected from the group consisting of: Sendai virus (SeV), human parainfluenza virus-1 (HPIV-1), human parainfluenza virus-3 (HPIV-3), phocine distemper virus (PDV), canine distemper virus (CDV), dolphin molbillivirus (DMV), peste-des-petits-ruminants virus (PDPR), measles virus (MV), rinderpest virus (RPV), Hendra virus (Hendra), and Nipah virus (Nipah).

The vectors used in the present invention include viruses belonging to Paramyxovirinae (including the genera Respirovirus, Rubulavirus, and Morbillivirus) or derivatives thereof, for example, those belonging to the genus Respirovirus (also referred to as the genus Paramyxovirus) or derivatives thereof. The derivatives include viruses that are genetically-modified or chemically-modified in a manner not to impair their gene-transferring ability. Examples of viruses of the genus Respirovirus applicable to this invention are human parainfluenza virus-1 (HPIV-1), human parainfluenza virus-3 (HPIV-3), bovine parainfluenza virus-3 (BPIV-3), Sendai virus (also referred to as murine parainfluenza virus-1), and simian parainfluenza virus-10 (SPIV-10).

The viral vectors of the present invention may be derived from natural strains, wild-type strains, mutant strains, laboratory-passaged strains, artificially constructed strains, or the like. The viral vectors of the present invention may be transmissible or non-transmissible. Herein, transmissibility means that when a viral vector infects a host cell, the virus is replicated in the cell to produce infectious virions. The viral vectors may be those having the same structure as that of a virus isolated from a natural source or modified artificially by genetic recombination. For example, such viral vectors may have mutations or deletions in some genes of a wild-type virus. Alternatively, it is possible to use incomplete viruses such as DI particles (J. Virol. 68:8413-8417, 1994). For example, it is also preferred to use viruses having a mutation or deletion in at least one of the genes encoding the viral envelope proteins or coat proteins. Such a viral vector can replicate its genome, for example, in infected cells, but it cannot form infectious virions. Such replication-deficient viral vectors are highly safe, because there is no risk of spreading the infection. For example, it is possible to use minus-strand RNA viral vectors that lack at least one of the genes encoding envelope proteins such as F, H, HN, M, and M1, or spike proteins, or an arbitrary combination of two or more, three or more, or four or more of them (WO 00/70055; WO 00/70070; Li, H.-O. et al., J. Virol. 74(14):6564-6569, 2000). When the genomic RNA encodes proteins required for genomic replication (for example, N, P, and L proteins), the genome can be amplified in infected cells. To produce defective viruses, for example, some genes are deleted from the viral genome, and deficient gene products or proteins capable of complementing them are exogenously supplied to virus-producing cells (WO 00/70055; WO 00/70070; Li, H.-O. et al., J. Virol. 74(14):6564-6569, 2000). There is also a known method for harvesting such viral vectors as non-infectious virions (VLP) without completely complementing the deficient viral proteins (WO 00/70070). Alternatively, by harvesting RNP (for example, RNP consisting of N, L, and P proteins, and genomic RNA), the vectors can be produced without complementing the envelope proteins.

Meanwhile, when enveloped virus-derived viral vectors are prepared, viral vectors that contain in their envelope proteins different from the original viral envelope proteins can be produced. Viruses containing a desired foreign envelope protein can be produced, for example, by expressing the protein in virus-producing cells at the time of virus production. Such proteins are not particularly limited, and it is possible to use any desired protein such as adhesion factor, ligand, or receptor that confers infectivity to mammalian cells. Specifically, for example, the proteins include vesicular stomatitis virus (VSV) G protein (VSV-G). The VSV-G protein may be derived from any VSV strain, and includes, but is not limited to, for example, VSV-G protein derived from the Indiana serotype strain (J. Virology 39:519-528, 1981).

The viral vectors of the present invention encode an Aβ antigen peptide in a form of fusion protein with AB5 toxin B subunit. Herein, the Aβ antigen peptide refers to an antigen peptide derived from Aβ. The peptide contains Aβ or an antigenic fragment thereof. The Aβ antigen peptide of the present invention includes, but is not limited to, the naturally occurring Aβ and antigenic fragments thereof (fragments having 6, 7, 8, 9, 10, 11, 12, 13, 14, 15, or more amino acids), and synthetic peptides resulting from adding a different amino acid sequence to them or linking them together in an arbitrary combination (Harlow, Antibodies: A laboratory Manual, 1998; Chapter 5 page 76). The preferred Aβ antigen peptides have one or more B cell epitopes of Aβ. The origin of Aβ is not particularly limited; however, Aβ peptides derived from human Aβ (Aβ40, Aβ42, Aβ43, etc.) are preferably used (as an example, the sequence of Aβ43 is shown in SEQ ID NO: 69). More specifically, the Aβ peptides include Aβ1-39, Aβ1-40, Aβ1-41, Aβ1-42, Aβ1-43, Aβ17-40, Aβ17-42, Aβ1-5, Aβ1-6, Aβ1-7, Aβ1-8, Aβ1-9, Aβ1-10, Aβ1-11, Aβ1-12, Aβ1-13, Aβ1-14, Aβ1-15, Aβ1-16, Aβ1-17, Aβ1-18, Aβ1-19, Aβ1-20, Aβ1-21, Aβ1-33, Aβ3-7, Aβ4-10, and peptides resulting from linking one or more copies of one or more of those listed above in an arbitrary combination, for example, in tandem (the number indicates position from the N terminus of Aβ) (Japanese Patent Application Kokai Publication No. (JP-A) 2005-21149 (unexamined, published Japanese patent application)). In addition, the anti-Aβ monoclonal antibodies 10D5 and 6C6, known for their ability to inhibit amyloid fibril formation and to protect neurons, have been reported to recognize a four amino acid-epitope corresponding to the 3rd to 6th amino acids of Aβ42 (EFRH) (Frenkel, D. et al., J. Neuroimmunol. 88:85-90, 1998; Frenkel, D. et al., J. Neuroimmunol. 95:136-142, 1999). The monoclonal antibody 508F that recognizes the same epitope also suppresses Aβ neurotoxicity (Frenkel, D. et al., J. Neuroimmunol. 106:23-31, 2000). Thus, a polypeptide of six to eight amino acids or more in the Aβ sequence including this sequence (EFRH) can be suitably used. Epitopes corresponding to cellular immunity are concentrated in the C-terminal region of Aβ. Accordingly, humoral immunity can be made relatively dominant over cellular immunity by expressing a fragment, which comprises an N-terminal fragment such as Aβ1-21, but not sequences near to the C-terminal as Aβ22-43.

The particularly preferred Aβ peptides include peptides containing one or more copies of a fragment from positions 1 to 3 up to positions 10 to 20 relative to the N terminus in the naturally occurring Aβ (Aβ1-10 to Aβ1-20, Aβ2-10 to Aβ2-20, or Aβ3-10 to Aβ3-20). Specifically, the peptides include those containing one or more copies of the sequence of Aβ1-10, Aβ1-11, Aβ1-12, Aβ1-13, Aβ1-14, Aβ1-15, Aβ1-16, Aβ1-17, Aβ1-18, Aβ1-19, Aβ1-20, Aβ2-10, Aβ2-11, Aβ2-12, Aβ2-13, Aβ2-14, Aβ2-15, Aβ2-16, Aβ2-17, Aβ2-18, Aβ2-19, Aβ2-20, Aβ3-10, Aβ3-11, Aβ3-12, Aβ3-13, Aβ3-14, Aβ3-15, Aβ3-16, Aβ3-17, Aβ3-18, Aβ3-19, or Aβ3-20. Since most of the B cell epitopes of Aβ42 are localized in the region from positions 1 to 15 relative to the N terminus (Cribbs, D. H. et al., Int. Immunol. 15(4):505-14, 2003), for example, a polypeptide comprising Aβ1-15 (positions 1 to 15 in SEQ ID NO: 1) or a fragment thereof can be preferably used as an Aβ peptide. The fragment length is not limited as long as the fragment contains such epitopes. The length may be, for example, 6, 7, 8, 9, 10, or longer. For example, fragments containing Aβ3-6 (EFRH) are preferred. It is more preferred to use peptides containing one or more copies of Aβ1-15 or fragment thereof, for example, peptides containing 1 to 12 copies, preferably 2 to 10 copies, and more preferably 2 to 8 copies, 3 to 8 copies, or 4 to 8 copies of Aβ1-15 or fragment thereof. Multiple copies of Aβ1-15 or fragment thereof are preferably linked in tandem via a linker. The linker sequence is not particularly limited; however, it may be an amino acid sequence, for example, of 1 to 15 amino acids, preferably 1 to 8 amino acids, for example, 1 to 6 amino acids. Specifically, the linker includes, but is not limited to, for example, K (lysine), KK, KKK, GP (glycine-proline), GPGP, GGS (glycine-glycine-serine), GGGS, GGGGS, repeats thereof, and various combinations thereof.

Herein, the AB5 toxin refers to a toxin shared by many pathogenic bacteria, which consists of a single copy of A subunit and five copies of B subunit (Merritt E and Hol W. “AB5 toxins”, Curr Opin Struct Biol 5(2):165-71, 1995; Lencer W and Saslowsky D “Raft trafficking of AB5 subunit bacterial toxins”, Biochim Biophys Acta 1746(3):314-21, 2005). Such AB5 toxin includes, for example, enterotoxin of Campylobacter jejuni, cholera toxin (Vibrio cholerae), heat-labile enterotoxin (for example, LT and LT-II) (Escherichia coli), pertussis toxin (Bordetella pertussis), shiga toxin (Shigella dysenteriae), and shiga-like toxin and verotoxin produced by other enterohemorrhagic bacteria. In general, the A subunit is responsible for the toxicity of these toxins, while the B subunit forms a pentamer involved in bacterial adhesion to cells.

The particularly preferred AB5 toxin of the present invention includes cholera toxin and E. coli heat-labile enterotoxin, and they are structurally and functionally similar to each other (Hovey B T et al., J Mol Biol., 285(3):1169-78, 1999; Ricci S. et al., Infect Immun. 68(2):760-766, 2000; Tinker J. K. et al., Infect Immun. 73(6):3627-3635, 2005). Specifically, the B subunit of cholera toxin or E. coli heat-labile enterotoxin includes proteins comprising a protein of accession numbers ZP_{—}01954889.1, ZP_{—}01976878.1, NP_{—}231099.1, P13811.1, ABV01319.1, or P32890, protein of SEQ ID NO: 14, or the mature form thereof (lacking the N terminal 21 amino acids; for example, amino acids of positions 22 to 124). Nucleotide sequences encoding such a protein include those comprising the nucleotide sequence of NZ_AAWE01000267.1, NC_{—}002505.1, M17874.1, EU113246.1, or M17873.1, or the sequence encoding a mature protein thereof (for example, lacking the 5′-most 63 nucleotides). The preferred AB5 toxin B subunit of the present invention includes those comprising an amino acid sequence described above or an amino acid sequence encoded by a nucleotide sequence described above, and those exhibiting high similarity to an above-described amino acid sequence.

Such AB5 toxin B subunit may comprise not only a naturally occurring sequence but also mutations. It is possible to use a B subunit comprising an amino acid sequence, for example, with an addition, deletion, substitution, and/or insertion of one or a small number of amino acids (for example, several amino acids, 3 amino acids or less, 5 amino acids or less, 10 amino acids or less, 15 amino acids or less, or 20 amino acids or less), as long as it does not significantly reduce the expression level of the fusion protein, the ability to induce anti-Aβ antibody, and/or the effect to alleviate at least one symptom of Alzheimer's disease, as compared to when using the naturally-occurring B subunit. It is also possible to use polypeptides with an N- and/or C-terminal amino acid deletion or addition of one to several residues (for example, 2, 3, 4, 5, 6, 10, 15, or 20 residues) and polypeptides with an amino acid substitution of one to several residues (for example, 2, 3, 4, 5, 6, 10, 15, or 20 residues). Usable variants include, for example, fragments, analogues, derivatives of a naturally occurring protein, and fusion proteins of a naturally occurring protein with other polypeptides (for example, those additionally containing a heterologous signal peptide or antibody fragment). Specifically, polypeptides that comprise a sequence with a substitution, deletion, and/or addition of one or more amino acids in the amino acid sequence of a wild-type B subunit, and have an activity comparable to or stronger than the activity of the wild-type B subunit to increase the expression level of the fusion protein with Aβ antigen peptide, induction of anti-Aβ antibody, and/or effect of alleviating at least one symptom of Alzheimer's disease, as compared to when the Aβ antigen peptide is expressed alone, can be used as an AB5 toxin B subunit of the present invention. Such modified AB5 toxin B subunits preferably retain the activity of forming a pentamer. When a fragment of a wild-type protein is used, it typically comprises a continuous region of 70% or more, preferably 80% or more, and more preferably 90% or more (or 95% or more) of the wild-type polypeptide (in its mature form when it is a secretory protein).

Amino acid sequence variants can be prepared, for example, by introducing mutations into DNAs that encode naturally occurring polypeptides (Walker and Gaastra, eds. (1983) Techniques in Molecular Biology (MacMillan Publishing Company, New York); Kunkel Proc. Natl. Acad. Sci. USA 82:488-492, 1985; Kunkel et al., Methods Enzymol. 154:367-382, 1987; Sambrook et al. (1989) Molecular Cloning: A Laboratory Manual (Cold Spring Harbor Laboratory Press, Plainview, N.Y.); U.S. Pat. No. 4,873,192). Guidance for substituting amino acids without affecting the polypeptide's biological activity includes, for example, Dayhoff et al. (1978) in Atlas of Protein Sequence and Structure (Natl. Biomed. Res. Found., Washington D.C.)). AB5B includes, for example, the R192G mutant of E. coli enterotoxin LT (Lemere et al., Neurobiol. Aging 23:991-1000, 2002; Seabrook et al., Neurobiol. Aging 25:1141-1151, 2004; Seabrook et al., Vaccine 22:4075-7083, 2004).

The number of amino acids to be modified is not particularly limited; however, it is, for example, 30% or less of the total amino acids in the mature form of a naturally occurring polypeptide, preferably 25% or less, more preferably 20% or less, even more preferably 15% or less, and still more preferably 10% or less, 5% or less, 3% or less, or 1% or less, and for example, 15 amino acids or less, preferably 10 amino acids or less, even more preferably 8 amino acids or less, still more preferably 5 amino acids or less, and yet more preferably 3 amino acids or less. In amino acid substitution, the original activity of a protein is expected to be retained by substituting amino acids having side chains with similar properties. Herein, this type of substitution is referred to as conservative substitution. Conservative substitutions include substitutions between amino acids within the same group, such as basic amino acids (for example, lysine, arginine, and histidine), acidic amino acids (for example, aspartic acid and glutamic acid), non-charged polar amino acids (for example, glycine, asparagine, glutamine, serine, threonine, tyrosine, and cysteine), nonpolar amino acids (for example, alanine, valine, leucine, isoleucine, proline, phenylalanine, methionine, and tryptophan), β-branched amino acids (for example, threonine, valine, and isoleucine), and aromatic amino acids (for example, tyrosine, phenylalanine, tryptophan, and histidine). Conservative substitutions also include, for example, substitutions between amino acids that give a positive score (for example, +1 or higher, +2 or higher, +3 or higher, or +4 or higher score) in the BLOSUM62 substitution matrix (S. Henikoff and J. G. Henikoff, Proc. Acad. Natl. Sci. USA 89:10915-10919, 1992).

Modified proteins exhibit high homology to the amino acid sequence of a wild-type protein. High homology amino acid sequences include those with an identity of, for example, 70% or higher, 75% or higher, 80% or higher, 85% or higher, 90% or higher, 93% or higher, 95% or higher, or 96% or higher. The amino acid sequence identity can be determined, for example, using the BLASTP program (Altschul, S. F. et al., J. Mol. Biol. 215:403-410, 1990). For example, the search can be carried out on the BLAST web page of National Center for Biotechnology Information (NCBI) using default parameters (Altschul S. F. et al., Nature Genet. 3:266-272, 1993; Madden, T. L. et al., Meth. Enzymol. 266:131-141, 1996; Altschul S. F. et al., Nucleic Acids Res. 25:3389-3402, 1997; Zhang J. & Madden T. L., Genome Res. 7:649-656, 1997). Sequence identity can be determined, for example, by comparing two sequences using the blast2sequences program to prepare an alignment of the two sequences (Tatiana A et al., FEMS Microbiol Lett. 174:247-250, 1999). Gaps are treated in the same way as mismatches. For example, an identity score is calculated over the entire amino acid sequence within the aligned region of a naturally occurring protein (its mature form after secretion). Specifically, sequence identity is determined by calculating the ratio of the number of identical amino acids to the total number of amino acids in a wild-type protein (the mature form in the case of a secretory protein).

Meanwhile, the AB5 toxin B subunit includes proteins encoded by nucleic acids that hybridize under stringent conditions to the entire or a portion of the coding region of a gene encoding a wild-type protein, and have an activity comparable to that of the wild-type protein (the expression level of a fusion protein with an Aβ antigen peptide, induction of anti-Aβ antibody, and/or alleviation of at least one symptom of Alzheimer's disease). The proteins preferably form a pentamer. For hybridization, a probe is prepared from either a nucleic acid comprising the coding sequence of the gene of a wild type protein or a complementary sequence thereof, or a nucleic acid targeted in the hybridization. The identification can be achieved by detecting whether the probe hybridizes to other nucleic acids. Stringent hybridization conditions include, for example, conditions where hybridization is carried out using a solution containing 5×SSC, 7% (W/V) SDS, 100 μg/ml denatured salmon sperm DNA, and 5×Denhardt's solution (1×Denhardt's solution contains 0.2% polyvinylpyrrolidone, 0.2% bovine serum albumin, and 0.2% Ficoll) at 50° C., preferably at 60° C., and more preferably at 65° C., and where post-hybridization washing is carried out with shaking for two hours at the same temperature as for the hybridization using 2×SSC, preferably 1×SSC, more preferably 0.5×SSC, and still more preferably 0.1×SSC.

The signal peptide (typically, the first 21 amino acids) of an original AB5 toxin B subunit may be fused intactly with a recombinant protein. Alternatively, the signal peptide may be removed or, for example, a signal peptide of a protein derived from eukaryotic cells may be attached to the N terminus or replaced with the original signal peptide (see Examples). Specifically, it is possible to use the signal sequence of a desired secretory protein such as immunoglobulin kappa light chain, interleukin (IL)-2, tissue plasminogen activator (tPA), or amyloid precursor protein (APP); however, such signal sequences are not limited to these examples (refer to the signal sequences of accession Nos. NP_{—}958817 and NM_{—}201414).

Furthermore, the fusion proteins may also contain tags, linkers, and spacers. For example, the Aβ antigen peptide and AB5 toxin B subunit may be linked together directly or via a linker (or spacer). The sequence of linker/spacer is not particularly limited; however, the sequence may consist of, for example, 1 to 15 amino acids, preferably 1 to 8 amino acids, for example, 2 to 6 amino acids, for example, about 4 amino acids. Specifically, the sequence includes, but is not limited to, KK (lysine-lysine), GP (glycine-proline), GPGP (glycine-proline-glycine-proline), GGS (glycine-glycine-serine), GGGS, GGGGS, repeats thereof, and various combinations thereof. In general, the Aβ peptide and AB5B are fused together so that AB5B is located on the N terminal side of the Aβ antigen peptide, i.e., the Aβ antigen peptide is located on the C terminal side of AB5B.

When a gene of a fusion protein is expressed from a minus-strand RNA viral vector, for example, expression levels of the gene can be controlled depending on the type of a transcriptional initiation sequence added in the upstream (to the 3′-side of a minus strand) of the gene (WO 01/18223). Expression levels can also be controlled depending on the position at which the foreign gene is inserted in the genome: the closer the inserting position is to 3′-end of a minus strand, the higher the expression level; the closer the inserting position is to 5′-end, the lower the expression level. Thus, the position for inserting a fusion protein-encoding gene can be appropriately adjusted to obtain a desired expression level of fusion protein, or to achieve the most suitable combination with viral protein-encoding genes in the vicinity of the inserted gene. In general, as it is thought to be advantageous to get high expression levels of a fusion protein of AB5B-Aβ antigen peptide, it is preferable to link a fusion protein-encoding nucleic acid to a high-efficiency transcriptional initiation sequence and thus to insert it near to 3′-end of the minus strand genome. Specifically, a fusion protein-encoding gene is inserted between 3′-leader region and a viral protein ORF closest to 3′-end. Alternatively, the gene may be inserted between an ORF of the viral gene closest to 3′-end and the next viral gene. In wild type Paramyxoviruses, the viral protein gene closest to 3′-end of the genome is N gene, and the second closest gene is P gene. Alternatively, when a high level expression of the introduced gene is undesirable, the level of gene expression from the viral vector can be suppressed in order to get an appropriate effect, for example, by inserting the foreign gene at a site in the vector as close as possible to 5′-side of the minus strand genome, or by selecting a low-efficiency transcriptional initiation sequence.

Furthermore, the vectors of the present invention may carry a different foreign gene at a site other than the insertion site of the gene encoding a fusion protein of AB5B-Aβ antigen peptide. Such foreign genes are not limited. The foreign genes may be, for example, marker genes for monitoring vector infection, genes for cytokines, hormones, receptors, or antibodies that regulate the immune system, or fragments thereof, or other genes. The vectors of the present invention enable gene transfer either by direct (in vivo) administration at a target site of the body or indirect (ex vivo) administration by introducing a vector into patient-derived cells or other cells, followed by injection of the cells at a target site. The vectors of the present invention can be used as an excellent means to achieve efficient induction of anti-Aβ antibody, treat Alzheimer's disease, or prevent or suppress the progression of the disease.

Recombinant RNA viral vectors may be reconstituted using known methods. Specifically, the vectors can be produced via the steps of: (a) introducing into cells or transcribing in cells the genomic RNA of RNA virus encoding a fusion protein between an AB5 toxin B subunit and an Aβ antigen peptide, or an RNA encoding a complementary strand thereof in the presence of viral proteins that constitute the RNP containing the viral genome RNA; and (b) harvesting the generated virus or RNP containing the genomic RNA. The “viral proteins that constitute the RNP” described above typically refer to proteins that, together with the viral genome RNA, form RNP and constitute a nucleocapsid. These are a group of proteins necessary for genome replication and gene expression, and in the minus-strand RNA virus they are typically N (nucleocapsid (also referred to as nucleoprotein (NP))), P (phospho), and L (large) proteins. Although these notations vary depending on viral species, corresponding groups of proteins are known to those skilled in the art (Anjeanette Robert et al., Virology 247:1-6, 1998).

Desired mammalian cells, avian cells, or the like can be used to produce the viral vectors. Specifically, such cells include, for example, culture cells such as monkey kidney-derived LLC-MK₂ cells (ATCC CCL-7), CV-1 cells (for example, ATCC CCL-70), and hamster kidney-derived BHK cells (for example, ATCC CCL-10), and human-derived cells. Alternatively, when the viruses can be amplified in hen eggs, the viral vector can be prepared on a large scale by infecting embryonated hen eggs with a viral vector obtained from the above-described hosts. Methods for manufacturing viral vectors using hen eggs have been already developed (Nakanishi, et al., ed. (1993), “State-of-the-Art Technology Protocol in Neuroscience Research III, Molecular Neuron Physiology”, Koseisha, Osaka, pp. 153-172). Specifically, for example, a fertilized egg is placed in an incubator, and cultured for 9 to 12 days at 37° C. to 38° C. to grow an embryo. After inoculation of the viral vector into the allantoic cavity, the egg is incubated for several days (for example, three days) to propagate the viral vector. Then, allantoic fluid containing the vector is collected from the egg. Virus vectors can be separated and purified from allantoic fluid by conventional methods (Tashiro, M., “Virus Experiment Protocol”, Nagai, Ishihama, ed., Medical View Co., Ltd., pp. 68-73 (1995)).

The viral proteins required for the particle formation may be expressed from the transcribed viral genome RNA or supplied in trans from a source other than the genomic RNA. For example, to reconstitute the minus-strand RNA viral vector, N, P, and L proteins can be supplied by introducing into cells plasmids or the like that express them. The transcribed genomic RNAs are allowed to replicate in the presence of these viral proteins, resulting in formation of functional RNPs or virions. In order to express viral proteins and RNA genome in cells, a vector linked with DNA encoding the proteins or genome downstream of an appropriate promoter is introduced into host cells. Such promoters include, for example, CMV promoter (Foecking, M. K. and Hofstetter H., Gene 45:101-105, 1986), retroviral LTR (Shinnik, T. M., Lerner, R. A. & Sutcliffe, Nature 293:543-548, 1981), EF1-α promoter, CAG promoter (Niwa, H. et al., Gene 108:193-199, 1991; JP-A (Kokai) H03-168087).

In the production of defective viruses in which the genes of envelope proteins or such have been deleted, the infectivity of produced viruses can be complemented by expressing the deleted proteins and/or other viral proteins or such that can complement the function in virus-producing cells (WO 00/70055; WO 00/70070; WO 03/025570; Li, H.-O. et al., J. Virol. 74(14):6564-6569, 2000). For example, the viruses may also be pseudotyped with envelope proteins of viruses of a different origin from the virus from which the viral vector genome is derived. Such an envelope protein used may be, for example, the G protein of vesicular stomatitis virus (VSV) (VSV-G) (J. Virology 39:519-528, 1981) (Hirata, T. et al., J. Virol. Methods, 104:125-133, 2002; Inoue, M. et al., J. Virol. 77:6419-6429, 2003; Inoue M. et al., J Gene Med. 6:1069-1081, 2004). As for minus-strand RNA viral vectors, genes to be deleted from the genome include, for example, genes of spike proteins such as F, HN, H, and G genes of envelope-lining proteins such as M, and any combinations thereof. Deletion of a spike protein gene is effective in rendering minus-strand RNA viral vectors nontransmissible, whereas deletion of the gene of an envelope-lining protein such as M protein is effective in disabling the particle formation from infected cells. For example, F gene-defective minus-strand RNA viral vectors (Li, H.-O. et al., J. Virol. 74:6564-6569, 2000), M gene-defective minus-strand RNA viral vectors (Inoue, M. et al., J. Virol. 77:6419-6429, 2003), and the like are preferably used. Moreover, greater safety would be assured with vectors defective in any combination of at least two of F, HN (or H) and M genes. For example, vectors lacking both M and F genes are nontransmissible and defective in particle formation while retaining high level infectivity and gene expression ability.

For instance, in an example of the production of F gene-defective recombinant viruses, a plasmid expressing a minus-strand RNA viral genome defective in F gene or a complementary strand thereof is transfected into host cells along with an expression vector expressing F protein and expression vectors for N, P, and L proteins. Alternatively, viruses can be more efficiently produced by using host cells in which the F gene has been incorporated into their chromosomes (WO 00/70070). In this case, a sequence-specific recombinase such as Cre/loxP and FLP/FRT and a target sequence thereof are preferably used so that the F gene can be inducibly expressed (see WO 00/70055; WO 00/70070; Hasan, M. K. et al., J. General Virology 78:2813-2820, 1997). Specifically, for example, the envelope protein genes are integrated into a vector having a recombinase target sequence, such as the Cre/loxP inducible expression plasmid pCALNdlw (Arai, T. et al., J. Virology 72:1115-1121, 1998). The expression is induced by, for example, infection with the adenovirus AxCANCre at an MOI of 3 to 5 (Saito et al., Nucl. Acids Res. 23:3816-3821, 1995; and Arai, T. et al., J. Virol. 72:1115-1121, 1998).

In the vectors of the present invention, any viral gene comprised may be altered from the wild-type gene, for example, to reduce the immunogenicity of viral proteins, or to enhance the efficiency of RNA transcription or replication. Specifically, for example, the transcriptional or replicational function of minus-strand RNA viral vector can be enhanced by altering at least one of the replication factor genes, N, P, and L. The HN protein, which is an envelope protein, has both hemagglutinin activity and neuraminidase activity. For example, the viral stability in blood can be enhanced by attenuating the hemagglutinin activity, and infectivity can be controlled by modifying the neuraminidase activity. The membrane fusion ability can be controlled by altering the F protein. Furthermore, for example, the antigen-presenting epitopes of the F protein or HN protein which may act as antigenic molecules on the cell surface can be analyzed, and this information can be used to prepare viral vectors that have a reduced antigenicity of these proteins. Furthermore, a temperature-sensitive mutation may be introduced into a viral gene to suppress release of secondarily released particles (or virus-like particles (VLPs)) (WO 2003/025570).

Specific examples of viral protein mutations include mutation of Glu at position 86 (E86) of the SeV P protein, substitution of another amino acid for Leu at position 511 (L511) of the SeV P protein, or substitution of homologous sites in the P protein of a different minus-strand RNA virus. Specific examples include substitution of Lys at position 86, and substitution of Phe at position 511. Regarding the L protein, examples include substitution of other amino acids for Asn at position 1197 (N1197) and/or for Lys at position 1795 (K1795) in the SeV L protein, or substitution of homologous sites in the L protein of another minus-strand RNA virus, and specific examples include substitution of Ser at position 1197, and substitution of Glu at 1795. Mutations of the P gene and L gene can significantly increase the effects of persistent infectivity, suppression of the release of secondary virions, and suppression of cytotoxicity. For example, the following mutations can be introduced: G69E, T116A, and A183S for the M gene; and A262T, G264, and K461G for the HN gene. However, mutations that can be introduced are not limited thereto (see WO 2003/025570).

The minus-strand RNA viruses, for example, can be produced by the following known methods: WO 97/16539; WO 97/16538; WO 00/70055; WO 00/70070; WO 01/18223; WO 03/025570; WO 2005/071092; WO 2006/137517; WO 2007/083644; WO 2008/007581; Hasan, M. K. et al., J. Gen. Virol. 78:2813-2820, 1997; Kato, A. et al., EMBO J. 16:578-587, 1997; Yu, D. et al., Genes Cells 2:457-466, 1997; Durbin, A. P. et al., Virology 235:323-332, 1997; Whelan, S. P. et al., Proc. Natl. Acad. Sci. USA 92:8388-8392, 1995; Schnell. M. J. et al., EMBO J. 13:4195-4203, 1994; Radecke, F. et al., EMBO J. 14:5773-5784, 1995; Lawson, N. D. et al., Proc. Natl. Acad. Sci. USA 92:4477-4481; Garcin, D. et al., EMBO J. 14:6087-6094, 1995; Kato, A. et al., Genes Cells 1:569-579, 1996; Baron, M. D. and Barrett, T., J. Virol. 71:1265-1271, 1997; Bridgen, A. and Elliott, R. M., Proc. Natl. Acad. Sci. USA 93:15400-15404, 1996; Tokusumi, T. et al., Virus Res. 86:33-38, 2002; Li, H.-O. et al., J. Virol. 74:6564-6569, 2000. Following these methods, minus-strand RNA viruses including parainfluenza virus, vesicular stomatitis virus, rabies virus, measles virus, rinderpest virus, Sendai virus, and the like can be reconstituted from DNA.

The methods for producing plus-strand RNA viruses include the following examples:

### (1) Coronavirus

Enjuanes L, Sola I, Alonso S, Escors D, Zuniga S.

Coronavirus reverse genetics and development of vectors for gene expression.

Curr Top Microbiol Immunol. 287:161-97, 2005. Review.

### (2) Togavirus

Yamanaka R, Zullo S A, Ramsey J, Onodera M, Tanaka R, Blaese M, Xanthopoulos K G.

Induction of therapeutic antitumor antiangiogenesis by intratumoral injection of genetically engineered endostatin-producing Semliki Forest virus.

Cancer Gene Ther. 2001 October; 8(10):796-802.
Datwyler D A, Eppenberger H M, Koller D, Bailey J E, Magyar J P.

Efficient gene delivery into adult cardiomyocytes by recombinant Sindbis virus.

J Mol Med. 1999 December; 77(12):859-64.

### (3) Picornavirus

Lee S G Kim D Y, Hyun B H, Bae Y S.

Novel design architecture for genetic stability of recombinant poliovirus: the manipulation of G/C contents and their distribution patterns increases the genetic stability of inserts in a poliovirus-based RPS-Vax vector system.

J Virol. 2002 February; 76(4):1649-62.
Mueller S, Wimmer E.

Expression of foreign proteins by poliovirus polyprotein fusion: analysis of genetic stability reveals rapid deletions and formation of cardioviruslike open reading frames.

J Virol. 1998 January; 72(1):20-31.

### (4) Flavivirus

Yun S I, Kim S Y, Rice C M, Lee Y M.

Development and application of a reverse genetics system for Japanese encephalitis virus.

J Virol. 2003 June; 77(11):6450-65.
Arroyo J, Guirakhoo F, Fenner S, Zhang Z X, Monath T P, Chambers T J.

Molecular basis for attenuation of neurovirulence of a yellow fever Virus/Japanese encephalitis virus chimera vaccine (ChimeriVax-JE).

J Virol. 2001 January; 75(2):934-42.

### (5) Reovirus

Roner M R, Joklik W K.

Reovirus reverse genetics: Incorporation of the CAT gene into the reovirus genome.

Proc Natl Acad Sci USA. 2001 Jul. 3; 98(14):8036-41. Epub 2001 Jun. 26.

For methods for propagating other RNA viruses and preparing recombinant viruses, see Experimental Virology, Detailed Discussion, 2nd Edition (Ed. Students' Association of The National Institute of Health; Maruzen, 1982).

The present invention also relates to compositions comprising a vector of the present invention. The compositions of the present invention include pharmaceuticals (pharmaceutical compositions) and reagents. Such compositions may be those comprising cells introduced with a vector of the present invention. In the production of the compositions of the present invention, the vector or cells may be combined with desired pharmaceutically acceptable carriers or vehicles, if needed. The “pharmaceutically acceptable carriers or vehicles” include desired solutions in which the vector or cells can be suspended, for example, phosphate-buffered saline (PBS), sodium chloride solutions, Ringer's solution, and culture media. When the vector is amplified using hen eggs or such, it may contain allantoic fluid. Furthermore, the compositions of the present invention may contain carriers or vehicles such as deionized water and an aqueous solution of 5% dextrose. In addition, the compositions may also contain vegetable oils, suspending agents, surfactants, stabilizers, biocidal agents, or such. It is also possible to add preservatives or other additives.

Furthermore, the compositions of the present invention can be combined with, as a carrier, an organic substance such as a biopolymer, or an inorganic substance such as hydroxyapatite; specifically, a collagen matrix, a polylactate polymer or copolymer, a polyethylene glycol polymer or copolymer, and a chemical derivative thereof, etc.

The vectors of the present invention, cells introduced with the vectors, and compositions comprising any one of them can be used for efficient expression of an Aβ antigen peptide and induction of anti-Aβ antibodies (humoral immunity against Aβ), and are useful in preventing and/or treating Alzheimer's disease. The vectors or compositions of the present invention can be administered directly or indirectly (for example, via cells) to individual organisms to induce anti-Aβ antibodies (humoral immunity against Aβ), or treat and/or prevent Alzheimer's disease. The present invention relates to methods for inducing an anti-Aβ antibody (humoral immunity against Aβ), which comprise the step of directly or indirectly administering a vector or composition of the present invention. Furthermore, the present invention provides methods for treating and/or preventing Alzheimer's disease, which comprise the step of directly or indirectly administering a vector or composition of the present invention. The present invention also relates to anti-Aβ antibody inducers and humoral immunity inducers against Aβ, both of which comprise a vector of the present invention, cells introduced with the vector, or a composition of the present invention. The present invention also provides the use of the vectors, cells, and compositions in inducing anti-Aβ antibodies or humoral immunity against Aβ. The present invention also provides use of the vectors of the present invention, cells introduced with the vectors, and compositions of the present invention in preventing and/or treating Alzheimer's disease. The present invention also provides use of the vectors of the present invention, cells introduced with the vectors, and compositions of the present invention in producing agents for inducing anti-Aβ antibodies (humoral immunity against Aβ). The present invention also provides use of the vectors of the present invention, cells introduced with the vectors, and compositions of the present invention in producing pharmaceutical agents for preventing and/or treating Alzheimer's disease. The present invention also relates to methods for producing agents for inducing anti-Aβ antibodies (humoral immunity against Aβ), which comprise the step of producing a composition comprising a vector of the present invention or a cell introduced with the vector in combination with a pharmaceutically acceptable carrier or vehicle. The present invention also relates to methods for producing pharmaceutical agents for treating and/or preventing Alzheimer's disease, which comprise the step of producing a composition comprising a vector of the present invention or a cell introduced with the vector in combination with a pharmaceutically acceptable carrier or vehicle. The present invention also relates to pharmaceutical agents for treating and/or preventing Alzheimer's disease, which comprises a vector of the present invention or cells introduced with the vector. The present invention also relates to pharmaceutical compositions for treating and/or preventing Alzheimer's disease, which comprise a composition of the present invention. The present invention also relates to the use of RNA virus genomic RNA encoding a fusion protein between an AB5 toxin B subunit and an Aβ peptide or a complementary strand thereof (antigenome RNA), or DNA encoding at least either of the two, in producing agents for inducing anti-Aβ antibodies (humoral immunity against Aβ). The present invention also relates to the use of RNA virus genomic RNA encoding a fusion protein between an AB5 toxin B subunit and an Aβ peptide or a complementary strand thereof (antigenome RNA), or DNA encoding at least either of the two, in producing pharmaceutical agents for preventing and/or treating Alzheimer's disease.

Herein, “treating Alzheimer's disease” means amelioration of at least one symptom of Alzheimer's disease, while “preventing Alzheimer's disease” means decrease in the incidence of at least one symptom of Alzheimer's disease, and/or reduction in the degree of the developed symptom. Such an effect may not necessarily be produced in an individual, and it may be a statistically significant effect. Such effects include, for example, reducing the blood Aβ level, accumulation of Aβ in brain tissues or the like, or number of senile plaques or ratio of the area of senile plaques in the brain tissue, or the like. The vectors and compositions of the present invention are useful as agents for suppressing accumulation of Aβ, in particular, accumulation of Aβ in brain tissues, blood, or such, as compared to without administration of the composition of the present invention. Furthermore, the vectors and compositions of the present invention are useful as agents for suppressing senile plaques, in particular, as agents for reducing the number and/or total area of senile plaques, as compared to without administration of the composition of the present invention.

As described above, the vectors of the present invention can be used by in vivo administration, or cell-mediated ex vivo administration. When the vectors are administered via cells, they are introduced into appropriate culture cells or cells harvested from animals as inoculation targets. When the vectors are introduced into cells in vitro (for example, in a test tube or dish), they are introduced in vitro (or ex vivo), for example, in a desired physiological aqueous solution such as culture medium, physiological saline, blood, plasma, serum, or body fluid, where the multiplicity of infection (MOI; the number of infectious viruses per cell) preferably ranges from 1 to 1000, more preferably 2 to 500, even more preferably 3 to 300, and still more preferably 5 to 100. The resulting cells can be inoculated directly or as cell homogenate (lysate). Such inoculation is preferably carried out using cells expressing an AB5B-Aβ antigen peptide fusion protein by a vector of the present invention. It is possible to express a fusion protein having a signal peptide by the vector to secrete to the outside of cells. To eliminate the growth potential, the cells may be treated with irradiation, ultraviolet radiation, a chemical agent, or such. A lysate of cells introduced with a vector can be prepared by using methods of lysing the cell membrane with surfactants, methods based on repeating freeze-thaw cycles, or such. The surfactants include non-ionic surfactants such as Triton X-100 and Nonidet P-40. More specifically, lysates of cells introduced a vector can be prepared by a procedure that lyses the cell membrane with a surfactant or a procedure that involves repetition of freeze-thaw cycles. Non-ionic surfactants, such as Triton X-100 and Nonidet P-40, can be applied within the concentration range of 0.1% to 1%. Such lysates can be obtained by, for example, collecting cell cluster with centrifugation after PBS washing and re-suspension in TNE buffer [25 mM Tris-HCl (pH7.5), 150 mM NaCl, 1 mM EDTA, 1% Nonidet P-40], and incubating the cell suspension allowed to stand on ice for 10 to 30 minutes. If the protein to be used as an antigen is soluble in the cytoplasm, the prepared lysates can be centrifuged (10,000×g for 10 minutes) to remove the unnecessary insoluble fraction as precipitates and the resulting supernatant can be used for immunization. If a lysate is administered at a site where surfactants are undesirable, the lysate may be prepared by re-suspending the cells in PBS after washing and disrupting the cells through five to six freeze-thaw cycles. Alternatively, the lysate may be prepared by sonication without using any surfactant from the beginning. The lysate may contain an RNA viral vector of the present invention and/or RNPs consisting of its genome and viral proteins.

Specific methods for introducing into cells or individuals cell lysates, RNPs that contain viral genomic RNAs, non-infectious viral particles (virus-like particles (VLPs)), or such include those known to those skilled in the art, such as methods that utilize calcium phosphate (Chen, C. & Okayama, H., BioTechniques 6:632-638, 1988; Chen, C. and Okayama, H., Mol. Cell. Biol. 7:2745, 1987), DEAE-dextran (Rosenthal, N., Methods Enzymol. 152:704-709, 1987), liposome or other various transfection reagents (Sambrook, J. et al. (1989) Molecular Cloning: A Laboratory Manual (Cold Spring Harbor Laboratory, Cold Spring Harbor, N.Y.)), or electroporation (Ausubel, F. et al. (1994) In Current Protocols in Molecular Biology (John Wiley and Sons, NY), Vol. 1, Ch. 5 and 9). Chloroquine may be added to the transfection to suppress the degradation in endosomes (Calos, M. P., Proc. Natl. Acad. Sci. USA 80:3015, 1983). Transfection reagents include, for example, DOTMA (Roche), Superfect Transfection Reagent (QIAGEN, Cat No. 301305), DOTAP, DOPE, DOSPER (Roche #1811169), TransIT-LT1 (Mirus, Product No. MIR 2300), CalPhos™ Mammalian Transfection Kit (Clontech #K2051-1), and CLONfectin™ (Clontech #8020-1). Enveloped viruses are known to incorporate host cell-derived proteins during virion formation, and such proteins can potentially cause antigenicity and cytotoxicity when introduced into cells (J. Biol. Chem. 272:16578-16584, 1997). It is thus advantageous to use RNPs without the envelope (WO 00/70055).

Moreover, virus RNPs can be directly produced in a cell by introducing into the cell an expression vector that transcribes viral genomic RNA encoding an AB5B-Aβ antigen peptide fusion protein and an expression vector that encodes viral proteins (the N, P, and L proteins) necessary for replicating the genomic RNAs. Cells into which a viral vector has been introduced may also be produced in such a manner.

The dose of the vector of the present invention varies depending on the type of disease, patient's weight, age, sex, and symptoms, purpose of administration, dosage form of the composition to be introduced, administration method, a gene to be introduced, and the like; however, those skilled in the art may appropriately determine the dose. The route of administration can be appropriately selected, and includes, for example, percutaneous, intranasal, transbronchial, intramuscular, intraperitoneal, and subcutaneous administration. However, the route is not limited to the above examples. In particular, preferred administration includes intramuscular administration, subcutaneous administration, intranasal administration (including administration using nasal drop, spray, catheter, or the like), intracutaneous administration to the palm or sole, direct administration into the spleen, and intraperitoneal administration. The number of inoculation sites may be one or more, for example, 2 to 15 sites. The inoculation dose may be appropriately adjusted depending on the subject animal for inoculation, inoculation site, frequency of inoculation, and the like. The vector is preferably administered at a dose in a range of about 10⁵ to 10¹¹ CIU/ml, more preferably about 10⁷ to 10⁹ CIU/ml, and still more preferably about 1×10⁸ to 5×10⁸ CIU/ml, in combination with a pharmaceutically acceptable carrier. When converted into a virus titer, the single dose for human is 1×10⁴ to 5×10¹¹ CIU (cell infectious unit), preferably 2×10⁵ to 2×10¹⁰ CIU. When the vector is inoculated via cells (ex vivo administration), for example, it can be introduced into human cells, preferably autologous cells. It is possible to use 1×10⁴ to 10⁹ cells, preferably 1×10⁵ to 10⁸ cells, or a lysate of the cells. When the vector is inoculated to a nonhuman animal, the dose can be converted from the above-described dose, for example, based on the body weight ratio or volume ratio (e.g., mean value) of the target site for administration between the animal of interest and human.

The administration frequency can be once or more as long as the side effects are within a clinically acceptable range. The same applies to the daily administration frequency. Although single administration can produce significant effects, the effects can be enhanced by introducing the vector twice or more. Alternatively, it is possible to administer a different Aβ antigen or a vector that expresses the antigen.

When the administration is carried out multiple times, it is possible to appropriately adjust the administration interval. The inoculation interval may be, for example, one week to several tens of months. More specifically, inoculation may be performed at 1- to 60-week intervals, at 2- to 60-week intervals, at 3- to 30-week intervals, at 4- to 20-week intervals, or at 5- to 10-week intervals. Furthermore, when the inoculation is carried out multiple times, for example, a vector of the present invention, a desired Aβ antigen peptide, a vector that expresses the peptide, and the like may be used in any combination, and booster immunization can be carried out via a desired inoculation route, such as intramuscular injection, intranasal administration, intracutaneous administration, or subcutaneous administration. When the inoculation is carried out multiple times, the vector of the present invention is administered at least once at any inoculation. Preferably, the vector of the present invention is administered at the first or second immunization; however, the vector may be administered at a different time besides the first and second immunizations. Furthermore, the vector of the present invention may be administered, for example, in combination with a purified or crude Aβ peptide, an AB5B-Aβ antigen peptide fusion protein, a desired vector encoding them, cells introduced with the vector or homogenate of the cells, or the like. In particular, the vectors of the present invention are preferably administered multiple times, or in combination with an AB5B-Aβ antigen peptide fusion protein. The fusion protein may be administered, for example, as a lysate of cells introduced with the vector of the present invention.

For example, it is preferable to inoculate the vector of the present invention at the first immunization and then inoculate the vector of the present invention or an Aβ antigen peptide (Aβ or a fragment thereof, a fusion protein containing it, etc.) at the second immunization. Alternatively, it is possible to inoculate an Aβ antigen peptide (Aβ or a fragment thereof, a fusion protein containing it, etc.) at the first immunization and then inoculate the vector of the present invention at the second immunization. The Aβ antigen peptide to be used for booster immunization includes, for example, those produced using a vector of the present invention, those produced using bacteria such as E. coli, those produced using animal cells, and synthetic peptides (see Examples).

The subject of administration includes desired vertebrates having an immune system (human and nonhuman vertebrates); the preferred subjects are birds and mammals, and more preferred subjects are mammals (human and nonhuman mammals). Specifically, such mammals include humans, nonhuman primates such as monkeys, rodents such as mice and rats, and all other mammals such as rabbits, goats, sheep, pigs, bovines, cats, and dogs. These animals are useful, for example, in efficiently producing an anti-Aβ antibody. In addition, Alzheimer's disease model animals are useful in assessing therapeutic effects of the vectors of the present invention. When the aim is to treat and/or prevent Alzheimer's disease, the subject of administration includes, for example, animals and patients having at least one factor responsible for Alzheimer's disease, at least one symptom of Alzheimer's disease, or a higher risk than healthy individuals, and tissues and cells derived from them, including, for example, individuals with Alzheimer's disease, individuals with increased Aβ level, individuals with enhanced Aβ deposition, individuals with an Alzheimer-type mutant gene, Alzheimer's disease model animals, and tissues and cells derived from them. For example, animals expressing Alzheimer-type mutants such as APP, PS-1, and/or PS-2 can be preferably used. Specifically, it is possible to use transgenic animals expressing APP with FAD mutations such as London mutations (V717I, etc.) and Sweden mutations (K670N and M671L), or the like (Hsiao K et al., Science. 274:99-102, 1996; Irizarry M et al., J Neuropath Exper Neurol. 56:965-973, 1997; Sturchler-Pierrat C et al., Proc Natl Acad Sci USA. 94(24):13287-13292, 1997; Proc Natl Acad Sci USA 92:2041-2045, 1995).

When a vector of the present invention is administered, a fusion protein containing an Aβ antigen peptide is expressed at a high level, resulting in the induction of humoral immunity against Aβ (anti-Aβ antibody). The immunity is expected to ameliorate at least one symptom of Alzheimer's disease. Symptoms of Alzheimer's disease include, for example, accumulation and/or deposition of Aβ in brain tissues or blood, an increase in the number of senile plaques or percent area of senile plaques in the brain, enhanced microglial activity, microglial infiltration and/or accumulation in the brain, in particular, to senile plaques, intracerebral accumulation of substances that are activated upon inflammation, for example, complements, and learning and/or memory impairment. In particular, administration of the vector of the present invention is expected to increase the blood level of anti-Aβ antibody and/or reduce Aβ in brain tissues. Anti-Aβ antibody by itself is known to produce a therapeutic effect against Alzheimer's disease, and thus the increase in the blood level of anti-Aβ antibody can serve as an indicator for the therapeutic effect.

Induction of humoral immunity against Aβ can be confirmed by detection of the anti-Aβ antibody in plasma. The antibody level can be measured with an ELISA (enzyme-linked immunosorbent assay) or Ouchterlony's method. An ELISA method is carried out, for example, by attaching an antigen to a microplate, preparing an antiserum and making a series of 2-fold dilutions of the prepared antiserum (starting solution 1:1000), and adding the diluted antiserum to the plate for antigen-antibody reaction to take place. Then, for color development, the antibody of the immunized animal is reacted with a xenogenic antibody, which has been enzymatically labeled with peroxidase and serves as a secondary antibody. The antibody titer can be calculated based on the dilution fold of the antibody when the absorbance is one half of the maximum color absorbance. Alternatively, in Ouchterlony's method, antigens and antibodies diffuse within an agar gel, and white precipitation lines are formed as a result of immunoprecipitation reactions. The precipitation lines can be used to determine the antibody titer, which is the dilution fold of the antiserum when the immunoprecipitation reaction occurs. Aβ level in the brain tissue can be determined, for example, using brain tissue extracts and a BioSource ELISA kit or the like.

Furthermore, the present invention relates to methods for assessing the preventive or therapeutic effect against Alzheimer's disease, which comprise the steps of: administering a composition comprising a vector of the present invention or cells introduced with the vector of the present invention to an individual; and detecting at least one symptom of Alzheimer's disease in the individual. The subject of administration includes individuals having at least one factor responsible for Alzheimer's disease, at least one symptom of Alzheimer's disease, or a higher risk than healthy individuals, for example, individuals with Alzheimer's disease, Alzheimer's disease model animals, individuals with increased Aβ level, individuals with enhanced Aβ deposition, individuals with an Alzheimer-type mutant gene, and individuals with at least one symptom of Alzheimer's disease or still not developing but with a higher incidence of at least one symptom of Alzheimer's disease relative to normal individuals. A control for comparison may be individuals that have not been administered with a vector or composition of the present invention. When the vector or composition of the present invention is administered to individuals before onset of Alzheimer's disease, non-administered control individuals may be compared to see the effect of administration after at least one symptom of Alzheimer's disease is developed.

Symptoms of Alzheimer's disease to be assessed include enhancement of the intracerebral Aβ level, accumulation and deposition of Aβ, senile plaque formation, number of senile plaques, percent area of senile plaques in brain tissues, and learning and/or memory ability. Such methods can be used to monitor the therapeutic and preventive effects against Alzheimer's disease.

The effect of reducing Aβ deposition (senile plaques) can be determined, for example, by the following procedure: treating brain tissue sections with 70% formic acid and after inactivating endogenous peroxidase with 5% H₂O₂, reacting the sections with an anti-Aβ antibody (for example, 6E10 (Kim K S, et al. Neurosci. Res. Comm. 7:113, 1988)) and performing DAB stain using a peroxidase-labeled secondary antibody. After the staining, the area of Aβ accumulated site can be determined by microscopic observation. If the area fraction of the accumulated site decreased in comparison with that when a vector of the present invention was not administered, the level of Aβ deposition can be judged as reduced. Alternatively, senile plaques in a living subject can be observed using MRI after intravenous administration of such a compound as 1-fluoro-2,5-bis-(3-hydroxycarbonyl-4-hydroxy) styrylbenzene (FSB), which has an affinity to amyloid or such (Higuchi M et al., Nat. Neurosci. 8(4):527-33, 2005; Sato, K. et al., Eur. J. Med. Chem. 39:573, 2004; Klunk, W. E. et al., Ann. Neurol. 55(3):306-19, 2004). Effects of a vector of the present invention can be confirmed by such a non-invasive imaging technique for amyloids.

The present invention also relates to methods for assessing the immune response against Aβ, which comprise the steps of introducing a vector of the present invention, cells introduced with the vector, or a composition comprising either of the two to a subject with accumulation and/or deposition of Aβ or having a predisposition to accumulation and/or deposition of Aβ; and detecting anti-Aβ antibody in the subject. The subject having a predisposition to accumulation and/or deposition of Aβ means an individual that has a statistically higher incidence of Aβ accumulation and/or deposition than normal individuals. Such subjects include, for example, Alzheimer's disease model animals and individuals having Alzheimer-type mutant genes. The present invention also relates to methods for assaying Aβ accumulation and/or deposition, which comprise the steps of introducing a vector of the present invention, cells introduced with the vector, or a composition comprising either of the two to a subject with accumulation and/or deposition of Aβ or having a predisposition to accumulation and/or deposition of Aβ; and determining the level of Aβ accumulation and/or deposition in the subject. If needed, the effect of vector administration is assessed by comparing with non-administered individuals. These methods can be used to monitor the immune response against Aβ and/or the effect of reducing the accumulation/deposition of Aβ.

The present invention also relates to methods for inducing, detecting, or producing an anti-Aβ antibody, which comprise the step of administering to an individual a vector of the present invention, cell introduced with the vector, or a composition comprising either of the two. When an anti-Aβ antibody is detected, the methods additionally comprise the step of detecting the anti-Aβ antibody produced by the animal. Meanwhile, the methods for producing an anti-Aβ antibody additionally comprise the step of collecting an anti-Aβ antibody produced by the animal. Such individuals to be administered include those having an immune system, and are not limited to animals with Alzheimer's disease and animals with an increased incidence. For example, it is also possible to produce a human antibody against Aβ by administering a vector of the present invention to animals (mice) modified to produce the humanized antibody. The vector of the present invention enables efficient production of an anti-Aβ antibody because it can potently induce anti-Aβ antibodies. The prepared antibody is useful in detecting, isolating, purifying Aβ, and as a therapeutic agent (passive immunity agent) to suppress the accumulation of Aβ.

Furthermore, the present invention revealed that booster immunization using an RNA viral vector significantly increased the antibody titer (Example 11). It is assumed that high-level expression of an introduced gene is hardly achieved by multiple rounds of RNA viral vector administration. The reason is that such administration induces host immune response. However, the present inventors revealed that the antibody titer was significantly elevated when an RNA viral vector that expresses the antigen protein is inoculated after initial immunization with the RNA viral vector that expresses the antigen protein. This suggests that contrary to predictions based on the expression efficiency of an introduced gene, multiple rounds (two or more rounds) of administration of an RNA viral vector encoding an antigen protein are highly effective to increase the antibody titer against the antigen. Specifically, the present invention also relates to:

(1) a method for increasing the antibody titer against an antigen, which comprises the step of administering an RNA viral vector encoding the antigen protein two or more times;

(2) the method of (1), wherein the antigen is a fusion protein with an AB5 toxin B subunit;

(3) the method of (2), wherein the AB5 toxin B subunit is cholera toxin B (CTB);

(4) the method of any one of (1) to (3), wherein the antigen is an antigen of an infectious virus or microorganism, a cancer-associated antigen or an Alzheimer-associated antigen;

(5) the method of (4), wherein the antigen comprises an amyloid β antigen peptide;

(6) the method of (5), wherein the amyloid β antigen peptide comprises one or more copies of Aβ1-15 or a fragment thereof;

(7) the method of (5), wherein the amyloid β antigen peptide has a structure having one to eight copies of Aβ1-15 or a fragment thereof linked together;

(8) the method of (5), wherein the amyloid β antigen peptide has a structure having four to eight copies of Aβ1-15 linked together;

(9) the method of any one of (1) to (7), wherein the RNA viral vector is a minus-strand RNA viral vector;

(10) the method of (9), wherein the minus-strand RNA viral vector is a paramyxovirus vector;

(11) the method of (9), wherein the minus-strand RNA viral vector is a paramyxovirus vector;

(12) the method of any one of (1) to (11), wherein at least one administration is performed by intramuscular administration;

(13) the method of any one of (1) to (12), wherein at least one administration is performed by intranasal administration;

(14) the method of any one of (1) to (13), which is used to treat a disease;

(15) the method of (14), wherein the disease is infection, cancer, or Alzheimer's disease;

(16) a composition for increasing the antibody titer against an antigen by a method comprising the step of administering two or more times an RNA viral vector encoding the antigen protein, which comprises the RNA viral vector and a pharmaceutically acceptable carrier;

(17) the composition of (16), which is to be used in the method of any one of (1) to (15);

(18) the composition of (16) or (17), which is to be used to treat infection, cancer, or Alzheimer's disease;

(19) use of an RNA viral vector encoding an antigen protein, in producing an agent for increasing the antibody titer against the antigen by a method comprising the step of administering the vector two or more times;

(20) the use of (19) in producing an agent to be used in the method of any one of (1) to (15); and

(21) the use of (19) or (20) in producing an agent to be used to treat infection, cancer, or Alzheimer's disease.

The vector administration route, dose, interval, and the like may be appropriately selected, and are specifically described herein as an example. The antigen is not particularly limited, and includes those derived from infectious pathogenic microorganisms, viruses, and the like, cancer antigens, and antigens of Alzheimer's disease (Aβ and fragments thereof). The number of administration rounds may be two times, three times, four times, or more. Furthermore, it is possible to administer in multiple rounds of administration vectors that encode antigens not strictly identical to one another, as long as they share at least an antigen portion. For example, a vector encoding an antigen protein fused with an AB5 toxin B subunit may be administered at the initial immunization, and then a vector encoding the antigen protein alone, non-fused to the AB5 toxin B subunit, may be administered at the booster immunization, or vice versa. Furthermore, a different type of vector may be used at each administration, as long as they are RNA viral vectors. Paramyxovirus vectors are preferably used, and Sendai virus vectors are more preferred. The administration interval is not particularly limited, and may be appropriately adjusted to, for example, one week to six months, two weeks to four months, or three weeks to three months. Multiple rounds of administration may result in an increase in the antibody titer to, for example, 1.2 times or more, 1.3 times or more, 1.4 times or more, 1.5 times or more, 1.6 times or more, 1.7 times or more, 1.8 times or more, 1.9 times or more, or twice or more. The antibody titer can be determined by known methods such as ELISA. As described above, the vectors of the present invention can be used as pharmaceutical agents for preventing or treating Alzheimer's disease.

Meanwhile, the vectors of the present invention can be preferably used as virus-like particles (VLP), or used in the same way as conventional viral particles.

### EXAMPLES

Hereinbelow, the present invention is specifically described in the Examples; however, it is not to be construed as being limited thereto. All publications cited in the specification are incorporated herein as a part of the specification.

### Example 1

### Construction of SeV Vector Carrying the Aβ42 Gene

### (1) Preparation of Aβ42 Gene NotI Fragment

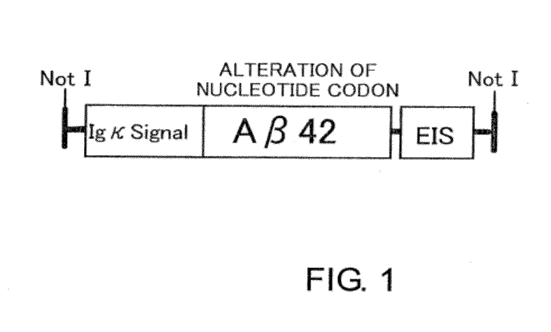

The Aβ42 gene was assembled by PCR using a number of primers that covered the entire length of the human amyloid β peptide sequence (1-42) (SEQ ID NO: 1). The nucleotide sequence of Aβ42 was optimized by taking the human codon usage into consideration. The resulting sequence had a structure where the Igκ secretory signal was attached to the N terminus and the transcription signal of Sendai virus was added to the C terminus (FIG. 1; SEQ ID NO: 2).

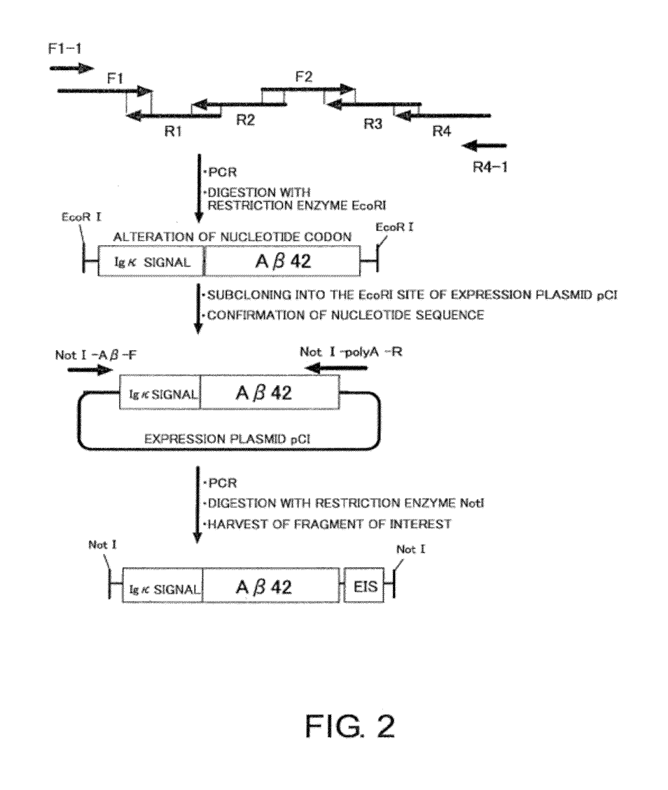

The construction method is shown in FIG. 2. First, the six types of long primers covering the entire region of Igκ signal and Aβ42, F1 (SEQ ID NO: 4), F2 (SEQ ID NO: 5), R1 (SEQ ID NO: 6), R2 (SEQ ID NO: 7), R3 (SEQ ID NO: 8), and R4 (SEQ ID NO: 9), were combined together. PCR was carried out using the primer mixture without a template. Then, the resulting PCR product was used as a template for the second round PCR and two primers F1-1 (SEQ ID NO: 10) and R4-1 (SEQ ID NO: 11), each having a restriction enzyme EcoRI recognition sequence, were used. The obtained PCR product was cleaved by restriction enzyme EcoRI and subcloned into the pCI expression plasmid (Promega) at the EcoRI site. A clone having the correct nucleotide sequence was selected by sequencing.

PCR was carried out using the selected plasmid as a template, as well as primer NotI-Aβ-F (SEQ ID NO: 12) with the addition of a NotI recognition sequence and primer NotI-polyA-R (SEQ ID NO: 13) with the addition of a Sendai virus transcription signal and NotI recognition sequence. The resulting PCR product was digested with the restriction enzyme NotI to give the Aβ42 NotI fragment of interest (FIG. 1; SEQ ID NO: 2).

(2) Construction of Sendai Virus cDNA Carrying the Aβ42 Gene

A cDNA of the F gene-deficient SeV vector (WO 00/70070) (pSeV18+NotI/ΔF) was digested with NotI, and the Aβ42 NotI fragment was inserted at the NotI site to construct F gene-deficient SeV cDNA carrying the Aβ42 gene (pSeV18+Aβ42/ΔF).

### Example 2

### Construction of SeV Vector Carrying a Fusion Gene of Aβ42 and CTB (CTB-Aβ42)

### (1) Preparation of CTB-Aβ42 Gene NotI Fragment

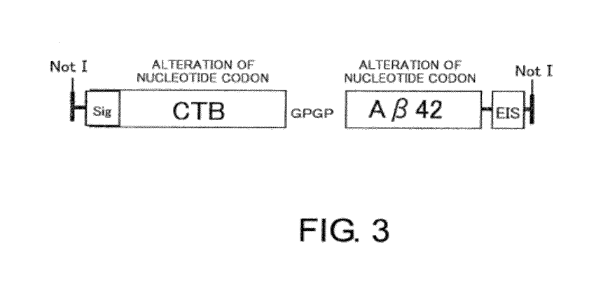

The CTB-Aβ42 NotI fragment had a structure in which a cholera toxin B subunit sequence (SEQ ID NO: 14) containing a secretory signal is linked via a GPGP amino acid linker to the N terminus of the human amyloid β sequence (1-42) and a Sendai virus transcription signal is attached to its C terminus (FIG. 3; SEQ ID NO: 15). To improve the expression efficiency, the nucleotide sequences of CTB and Aβ were altered according to the human codon usage without altering their amino acid sequences.

This gene was constructed by synthesizing its entire length by PCR using long primers that fully covered the gene. Specifically, eight types of long primers that covered the entire length of the CTB-Aβ region were synthesized: CTB-AβF-1 (SEQ ID NO: 17), F-2 (SEQ ID NO: 18), F-3 (SEQ ID NO: 19), F-4 (SEQ ID NO: 20), R-1 (SEQ ID NO: 21), R-2 (SEQ ID NO: 22), R-3 (SEQ ID NO: 23), and R-4 (SEQ ID NO: 24). By using a mixture of these eight types of primers, PCR was carried out to prepare a corresponding fragment from the N terminus to Aβ42. To prepare the C-terminal fragment containing the Sendai virus transcription signal, PCR was carried out using the pCI plasmid (Promega) as a template and two types of primers, CTB-Aβ F1-2 (SEQ ID NO: 25) and CTB-Aβ R5-2 (SEQ ID NO: 26). The PCR yielded a PCR fragment that covered the entire length of CTB-Aβ. The PCR fragment was subcloned into the pGEM-T Easy plasmid (Promega) by TA cloning. After the nucleotide sequence was confirmed, the plasmid was amplified and digested with restriction enzyme NotI to prepare a CTB-Aβ42 NotI fragment of interest (SEQ ID NO: 15).

(2) Construction of Sendai Virus cDNA Carrying the CTB-Aβ42 Gene

A cDNA of F gene-deficient SeV vector (WO 00/70070) (pSeV18+NotI/ΔF) was digested with NotI, and the CTB-Aβ42 NotI fragment was inserted into the NotI site to construct an F gene-deficient SeV cDNA carrying the CTB-Aβ42 gene (pSeV18+CTB-Aβ42/ΔF).

### Example 3

### Construction of SeV Vector Carrying a Fusion Gene of Aβ42 and IL-4

### (1) Preparation of a NotI Fragment of the Aβ42 and IL-4 Fusion Gene

The Aβ42 gene and mouse IL-4 were fused together by the assemble PCR method based on partial overlaps.

The Aβ42 gene was prepared from a plasmid containing an Aβ42 EcoRI fragment (Example 1; FIG. 2). Meanwhile, the mouse IL-4 gene (SEQ ID NO: 27) was prepared as a cDNA by the following procedure. mRNA was extracted from mouse (BALB/cA) spleen, and reverse transcribed using an IL-4-specific primer. The resulting cDNA was amplified by PCR and subcloned into a cloning plasmid. The resulting plasmid having mouse IL-4 cDNA as an insert was used in the construction.

Specifically, PCR was carried out using the mouse IL-4 plasmid as a template and two types of primers, NotI-IL4-F (SEQ ID NO: 29) and IL4-R (SEQ ID NO: 30). Meanwhile, using the Aβ42 plasmid as a template, PCR was carried out with primers Aβ42-F (SEQ ID NO: 31) and NotI-Aβ42-R (SEQ ID NO: 32), and IL-4 and Aβ42 PCR fragments were obtained. The primers IL4-R and Aβ42-F were designed to partially overlap with each other. Thus, the two genes were fused into a single fusion gene by PCR using a mixture of IL-4 and Aβ42 PCR fragments as template and the primers NotI-IL4-F and NotI-Aβ42-R. The resulting PCR fragment was subcloned into a cloning plasmid. After the nucleotide sequence was confirmed, the plasmid was cleaved with restriction enzyme NotI to prepare a NotI fragment of interest containing the fusion gene of Aβ42 and IL-4 (SEQ ID NO: 33).

(2) Construction of a Sendai Virus cDNA Carrying the Aβ42 Gene

A cDNA of F gene-deficient SeV vector (WO 00/70070) (pSeV18+NotI/ΔF) was digested with NotI. The mIL4-Aβ42 NotI fragment prepared as mentioned above was inserted into the NotI site to construct an F gene-deficient SeV cDNA carrying the Aβ42 gene (pSeV18+mIL4-Aβ42/ΔF).

### Example 4

### Reconstitution and Amplification of Sendai Virus Vector

Virus reconstitution and amplification were carried out according to the report of Li et al. (Li, H.-O. et al., J. Virology 74:6564-6569, 2000, WO 00/70070) and a modified method thereof (WO 2005/071092). Since the vectors used were F gene deficient, helper cells for the F protein, in which the F protein is expressed by the Cre/loxP expression induction system, were used. This system uses the pCALNdLw plasmid (Arai, T. et al., J. Virol. 72:1115-1121, 1988), which has been designed in a way that expression of gene products is induced by the Cre DNA recombinase. To express the inserted gene, a transformant with the plasmid is infected with a recombinant adenovirus expressing the Cre DNA recombinase (AxCANCre) by the method of Saito et al. (Saito, I. et al., Nucl. Acid. Res. 23:3816-3821, 1995; Arai, T. et al., S. Virol. 72:1115-1121, 1998).

F gene-deficient SeV vectors each carrying the CTB-mCRF, CTB-mET1, CTB-mPYY, CTB-mGLP2, mCRF, mET1, mPYY, mGLP2, Aβ42, CTB-Aβ42, or mIL4-Aβ42 gene (SeV18+CTB-mCRF/ΔF, SeV18+CTB-mET1/ΔF, SeV18+CTB-mPYY/ΔF, SeV18+CTB-mGLP2/ΔF, SeV18+mCRF/ΔF, SeV18+mET1/ΔF, SeV18+mPYY/ΔF, SeV18+mGLP2/ΔF, SeV18+Aβ42/ΔF, SeV18+CTB-Aβ42/ΔF, or SeV18+mIL4-Aβ42/ΔF, respectively) were prepared by the methods described above.

### Example 5

### Construction of SeV Vector Carrying a Fusion Gene of Aβ42 and PEDI

(1) Construction of SeV Vector cDNA Carrying the Aβ42-PEDI Fusion Gene

The PEDI gene was amplified by PCR using ten types of primers: PEDI-1F (SEQ ID NO: 35), PEDI-2R (SEQ ID NO: 36), PEDI-3F (SEQ ID NO: 37), PEDI-4R (SEQ ID NO: 38), PEDI-5F (SEQ ID NO: 39), PEDI-6R (SEQ ID NO: 40), PEDI-7F (SEQ ID NO: 41), PEDI-8R

(SEQ ID NO: 42), PEDI-9F (SEQ ID NO: 43), and PEDI-10R (SEQ ID NO: 44). The PEDI gene and Aβ42 were fused together and inserted into the SeV vector by the following procedure. Fragment 1 was prepared by PCR using the SeV vector carrying Aβ42 as a template with primers SeVF6 (SEQ ID NO: 45) and S-PEDI-C (SEQ ID NO: 46). Fragment 3 was prepared by PCR using the same template and primers PEDI-Ab-N (SEQ ID NO: 47) and SEVR280 (SEQ ID NO: 48). Fragment 2 was prepared by PCR using the PEDI gene as a template and primers PEDI-N (SEQ ID NO: 49) and PEDI-C (SEQ ID NO: 50). Using these fragments as a template, overlap PCR was carried out to prepare the PEDI-Aβ42 NotI fragment (SEQ ID NO: 51). The resulting fragment was inserted into the NotI site of pSeV18+/ΔF to prepare a cDNA of F-deficient SeV vector carrying the PEDI-Aβ42 fusion gene.

### (2) Reconstitution of an F-Deficient SeV Vector Carrying PEDI-Aβ42

An SeV vector carrying the PEDI-Aβ42 gene (SeV18+PEDI-Aβ42/ΔF) was reconstituted in the same way as described in Example 4 according to the method of Li et al. (Li, H.-O. et al., J. Virology 74:6564-6569, 2000; WO 00/70070) and its modified method (WO 2005/071092).

### Example 6

### Comparison of the Effects of CTB Fusion, PEDI Fusion, and IL-4 Fusion on Aβ42 Expression: Comparison of the Expression Abilities of SeV Vector for CTB-Aβ42 Fusion Protein Expression, SeV Vector for PEDI-Aβ42 Fusion Protein Expression, and SeV Vector for IL-4-A042 Fusion Protein Expression

(1) BHK21 Cells were Infected with SeV18+Aβ42/ΔF, SeV18+IL-4-Aβ42/ΔF, SeV18+PEDI-Aβ42/ΔF, and SeV18+CTB-Aβ42/ΔF to assess the level of Aβ42 antigen.

BHK21 cells were plated in collagen-coated 6-well plates at 1×10⁶ cells/well, and infected with each SeV vector diluted to an MOI of 10 with serum-free medium (VPSFM). GMEM containing 10% FBS was added to the plates after one hour. The medium was replaced with serum-free medium (VPSFM) 24 hours later, and the cells and culture supernatants were harvested after 48 hours to prepare cell lysates.

### (2) Quantitation by ELISA

Aβ42 was quantified using a human Aβ42 ELISA kit (Wako Pure Chemical Industries). The expression level was determined by absorbance (O.D. 450) measurement using a plate reader.

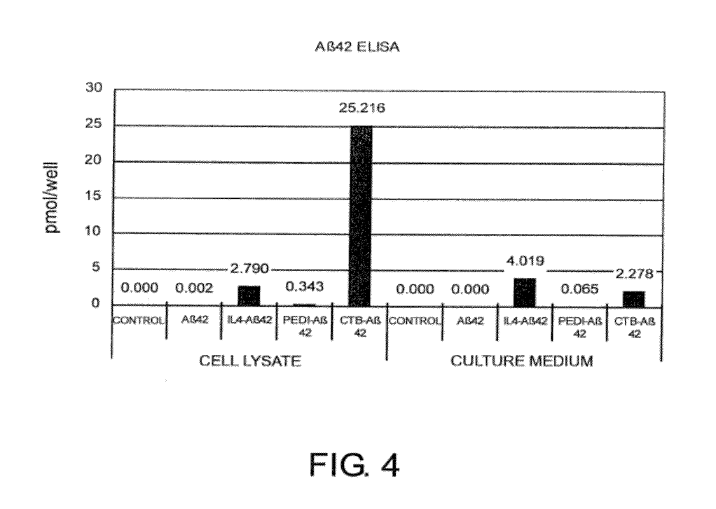

The result is shown in FIG. 4. The expression levels of IL-4-Aβ42, PEDI-Aβ42, and CTB-Aβ42 were increased, while Aβ42 was expressed at a negligible level. Each intracellular expression level was 1395 times, 171.5 times, or 12608 times that of kkAβ42.

### Example 7

### Effect of CTB Fusion on Aβ42 Expression: Comparison of the Expression Abilities Between SeV Vector to Express Aβ42 Alone and SeV Vector to Express CTB-Aβ42 Fusion Protein

Confluent BHK-21 cells (plated at 3×10⁵ cells/well in 12-well plates) plated on the previous day were infected at an MOI of 10 with an SeV vector carrying Aβ42 alone or an SeV vector carrying CTB-Aβ42, and then cultured in the VP-SFM medium (1 ml/well) at 37° C. under 5% CO₂. The culture supernatants were collected and cell lysates were prepared on day 4. The culture supernatants were concentrated ten-fold by acetone precipitation, and samples were prepared from them using 1×SDS Loading Buffer. The cell lysates were prepared in 150 μl/well using 1×SDS Loading Buffer. The prepared culture supernatants and cell lysates were heated at 98° C. for 10 minutes. Then, SDS-PAGE (using 15% Wako gel) and Western blotting (using antibody 6E10) were carried out, and proteins were quantified using the Aβ42 peptide as control at 1, 0.5, 0.25, and 0.125 ng/lane. With the SeV vector carrying Aβ42 alone, the Aβ expression level was only 4.4 ng/well or 7.2×10⁻³ ng/well in the cell lysate or supernatant, respectively. On the other hand, with the SeV vector carrying Aβ42-CTB fusion gene, the Aβ expression level was 2500 ng/well and 200 ng/well in the cell lysate and supernatant, respectively. That is, the Aβ expression level was greatly improved to be 568 and 27778 times higher in the lysate and supernatant, respectively.

### Example 8

### Construction of SeV Vectors Carrying the Aβ15-CTB Fusion Gene (CTB-A015) or a Fusion Gene of Tandem Aβ15 Repeats and CTB (CTB-Aβ15×2, CTB-Aβ15×4, or CTB-Aβ15×8)

### (1) Preparation of CTB-Aβ15 Gene NotI Fragment

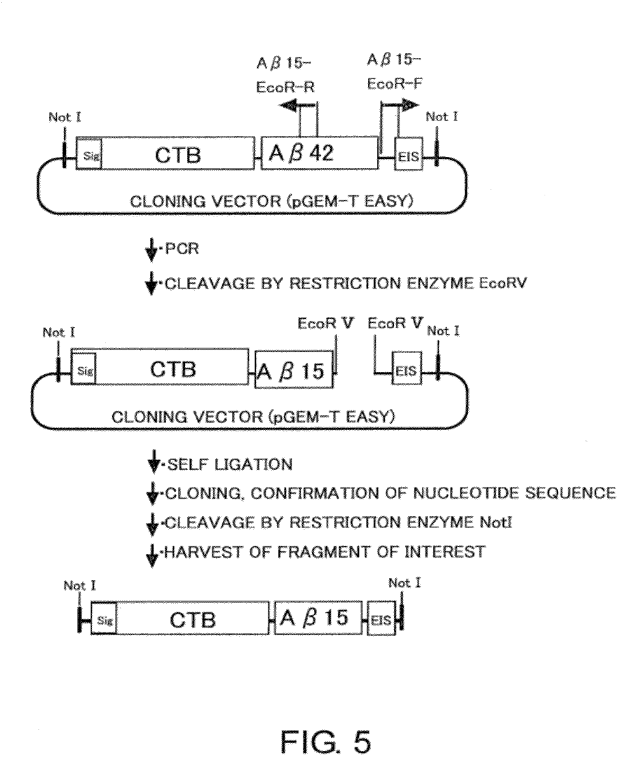

The CTB-Aβ15 gene NotI fragment was prepared based on the CTB-A042 gene (FIG. 5).

Inverse PCR was carried out using a plasmid containing the CTB-Aβ42 NotI fragment as a template and two types of primers Aβ15-EcoR-R (SEQ ID NO: 53) and Aβ15-EcoR-F (SEQ ID NO: 54), both of which have the addition of an EcoRV restriction site. The resulting PCR product was cleaved with restriction enzyme EcoRV. Then, the product was self ligated to prepare a plasmid containing the CTB-Aβ15 fragment. The plasmid was cleaved with restriction enzyme NotI to give the CTB-Aβ15 NotI fragment of interest (SEQ ID NO: 55).

(2) Preparation of NotI Fragments from a CTB-Tandem Aβ15 Gene (CTB-Aβ15×2, CTB-Aβ15×4, or CTB-Aβ15×8)

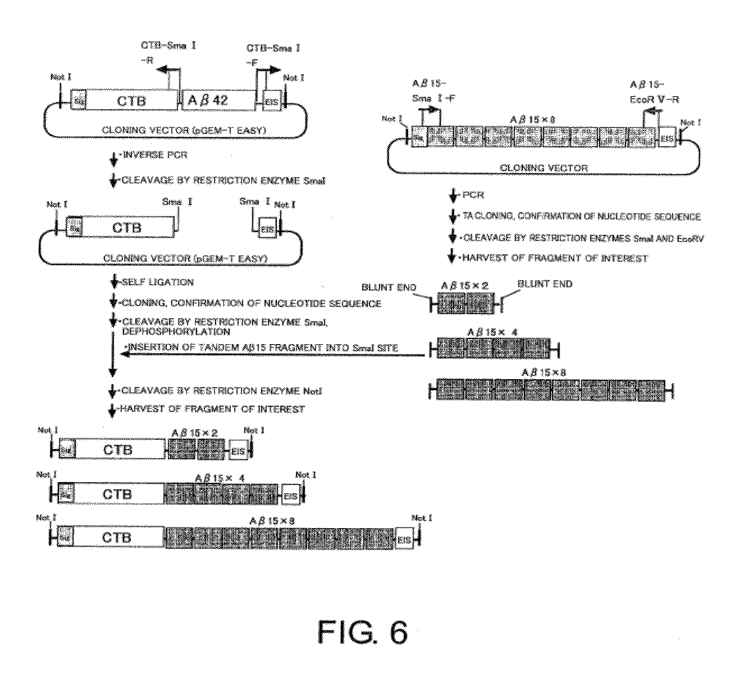

NotI fragments were prepared from a CTB-tandem Aβ15 gene using two types of genes (FIG. 6).

The method removed the Aβ42 region from the CTB-Aβ42-containing plasmid, and instead introduced a restriction site into the plasmid. A tandem Aβ15 fragment added with the restriction enzyme site was then inserted into that site of the plasmid by PCR.

Specifically, inverse PCR was carried out using as a template the plasmid containing the NotI fragment from CTB-Aβ42 (Example 2; SEQ ID NO:15), and two types of primers CTB-SmaI-R (SEQ ID NO: 57) and CTB-SmaI-F (SEQ ID NO: 58), both of which have the addition of a SmaI restriction enzyme site. The resulting PCR product was cleaved with SmaI, and then self ligated to give an Aβ42-deficient plasmid. Then, the tandem Aβ15 fragment was inserted into the SmaI site of the plasmid.

The tandem Aβ15 fragment was prepared based on a plasmid containing a tandem eight Aβ15 NotI fragment (SEQ ID NO: 59) by the following method. PCR was carried out using the plasmid as a template as well as two types of primers Aβ15-SmaI-F (SEQ ID NO: 61) and Aβ15-EcoRV-R (SEQ ID NO: 62), both of which have the addition of a restriction site. The PCR yielded PCR products that were different in the number of Aβ15 repeats. The products were cloned by TA cloning. After the nucleotide sequences were confirmed, blunt-ended tandem Aβ15 fragments were excised from the clones with two types of restriction enzymes SmaI and EcoRI. The fragments were each inserted into the SmaI site of the Aβ42-deficient plasmid. The resulting plasmids were amplified and cleaved with restriction enzyme NotI to give fragments of interest: CTB-Aβ15×2 NotI fragment (SEQ ID NO: 63), CTB-Aβ15×4 NotI fragment (SEQ ID NO: 65), and CTB-Aβ15×8 NotI fragment (SEQ ID NO: 67).

(3) Construction of Sendai Virus cDNAs Carrying CTB-Aβ15, CTB-Aβ15×2, CTB-Aβ15×4, or CTB-Aβ15×8 Gene

The cDNA of an F gene-deficient SeV vector (WO 00/70070) (pSeV18+NotI/ΔF) was digested with NotI, and CTB-Aβ15, CTB-Aβ15×2, CTB-Aβ15×4, and CTB-Aβ15×8 fragments were each inserted into the NotI site to construct F gene-deficient SeV cDNAs carrying CTB-Aβ15, CTB-Aβ15×2, CTB-Aβ15×4, or CTB-Aβ15×8 (pSeV18+CTB-Aβ15/ΔF, pSeV18+CTB-Aβ15×2/ΔF, pSeV18+CTB-Aβ15×4/ΔF, and pSeV18+CTB-Aβ15×8/ΔF).

### Example 9

### Comparison of the Ability to Express Aβ Peptide

### (1) Western Blotting

The constructed vectors were assessed by Western blotting for the infectivity and expression.

Homogenates and culture supernatants of cells infected with SeV vectors were mixed with an equal volume of SDS-PAGE sample buffer, and heated for thermal denaturation at 98° C. for five minutes. The mixtures were subjected to SDS-PAGE using 15% acrylamide gel, and then transferred onto PVDF membrane by the semi-dry blotting method. After blocking with 5% milk/TBS-T, the membrane was incubated with an anti-Aβ antibody (6E10) and then with an HRP-labeled anti-mouse IgG as the secondary antibody. Detection was carried out using chemiluminescent substrate SuperSignal West Femto with a CCD camera.

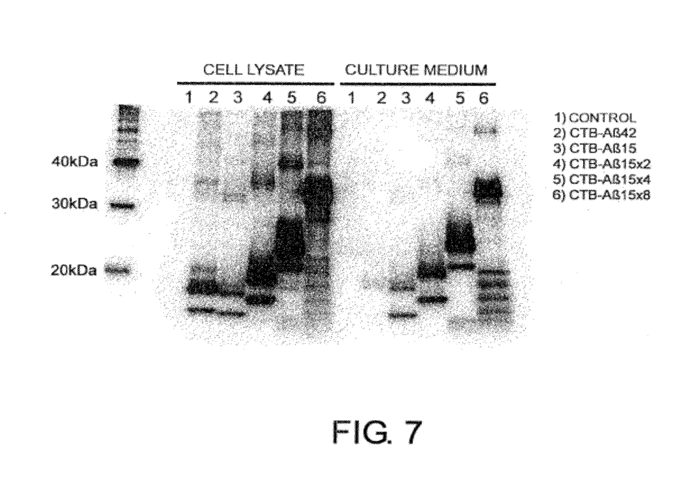

The result showed that CTB-Aβ42, CTB-Aβ15, CTB-Aβ15×2, CTB-Aβ15×4, and CTB-Aβ15×8 were expressed in the BHK cells and secreted to the media. It was also demonstrated that the expression level of CTB-Aβ15×8 was higher than that of CTB-Aβ42, and the quantity of CTB-Aβ15×8 secreted to the medium was considerably larger than that of CTB-Aβ42 (FIG. 7).

### (2) GM1-ELISA

The binding of CTB to GM1 was assessed using ganglioside GM1-immobilized plates.

Ganglioside GM1 (5 μg/ml) was immobilized onto each well of a 96-well plate (Nunc, MaxiSorp plate). After blocking with 20% BlockingOne (Nacalai Tesque), the culture supernatants of cells infected with the SeV vector were added (20- to 2,000,000-fold dilution) to the wells. Following incubation with the HRP-labeled antibody 6E10, detection was carried out using the TMB chromogenic reagent. The amount of binding was assessed by absorbance (O.D. 450) measurement using a plate reader.

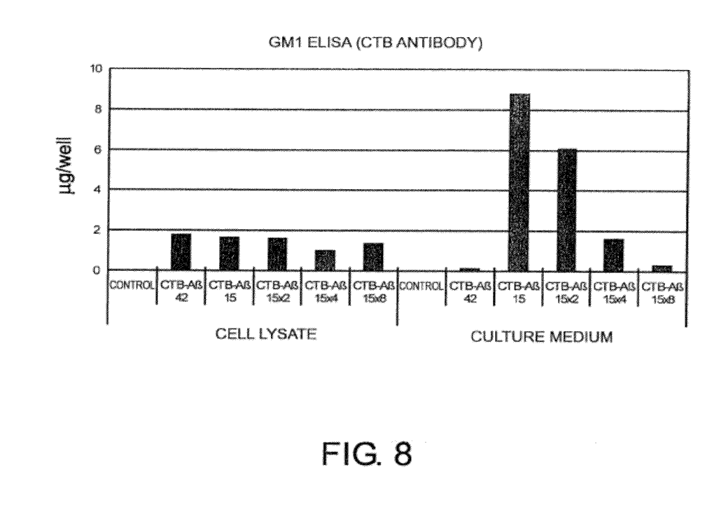

The result showed that CTB-Aβ42, CTB-415, CTB-Ali 15×2, CTB-Aβ15×4, and CTB-Aβ15×8 secreted to the media bound to GM1. The amount of CTB-Aβ15×8 binding was ten times that of CTB-Aβ42, while the amount of CTB-Aβ15 binding was 100 times that of CTB-Aβ15×8. This suggests that as the number of Aβ15 repeats increases, the GM1-binding ability is reduced (FIG. 8).

### Example 10

### Assessment of the Variously Constructed SeV Vectors for their Ability to Induce Anti-Aβ Antibody in Normal Mice

### (1) Normal Mice (Comparison, of Intramuscularly Administered CTB-Aβ42 and CTB-Aβ15×8)

SeV vectors carrying CTB-Aβ42, CTB-Aβ15×8, or GFP gene were each administered at a titer of 5×10⁷ CIU/head to C57BL/6N mice by intramuscular injection (right hind leg) to assess the antibody titer. 14 days after the treatment, blood was collected from the mice to determine the plasma level of anti-Aβ antibody. The Aβ1-42 peptide (5 μg/ml) was immobilized onto each well of a 96-well plate (Nunc, MaxiSorp plate). After blocking with 20% BlockingOne (Nacalai Tesque), mouse plasma (300- to 300,000-fold dilute) was added to the wells. Following incubation with a peroxidase-labeled mouse IgG antibody, detection was carried out using the TMB chromogenic reagent. The titer of the anti-Aβ antibodies was assessed by absorbance (O.D. 450) measurement using a plate reader. An anti-Aβ antibody (6E10) was used as the standard antibody.

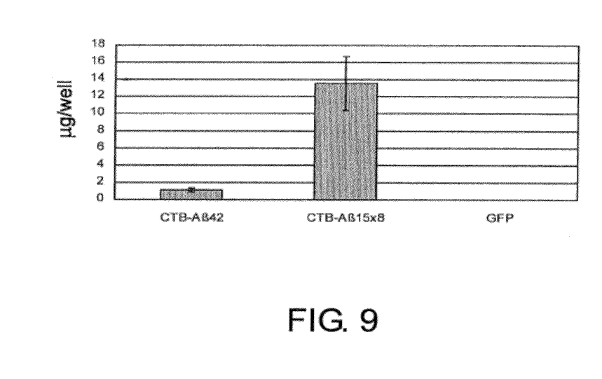

The result showed that the titer of the anti-Aβ antibodies was elevated in the CTB-Aβ42 gene administration group (n=6) and CTB-Aβ15×8 gene administration group (n=6), while it was not increased in the GFP gene administration group (n=6) as a control (FIG. 9). The antibody titer of the CTB-Aβ15×8 gene administration group was 12.23 times that of the CTB-Aβ42 gene administration group.

### (2) Normal Mice (Intramuscular, Intracutaneous, and Intranasal Administration)

An SeV vector carrying the CTB-Aβ15×8 gene was administered at a titer of 5×10⁶ or 5×10⁷ CIU/head intranasally, intracutaneously, and intramuscularly (right hind leg) to C57BL/6N mice, while an SeV vector carrying the GFP gene was administered at a titer of 5×10⁷ CIU/head intramuscularly (right hind leg) as the control group to assess the antibody titer. 14 days after the treatment, blood was collected from the mice to determine the plasma level of anti-Aβ antibody.

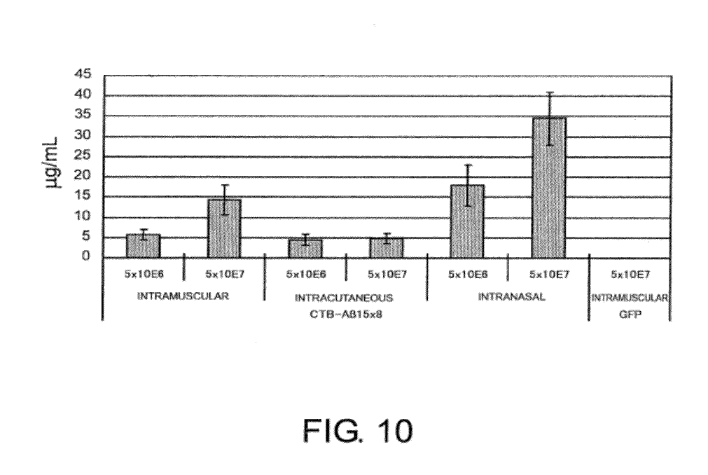

The result showed that the titer of anti-Aβ antibody was elevated in all administration groups except the control group. The antibody titer was lower in the intracutaneous administration group than in the other administration groups. The antibody titer was higher in the intranasal administration group when compared to the intramuscular administration group of the same titer (FIG. 10).

### (3) Normal Mice (Intranasal Administration)

SeV vectors carrying CTB-Aβ15, CTB-Aβ15×2, CTB-Aβ15×4, or CTB-Aβ15×8, and an SeV vector carrying the GFP gene as the control were intranasally administered at a titer of 5×10⁷ CIU/head to C57BL/6N mice to assess the antibody titer.

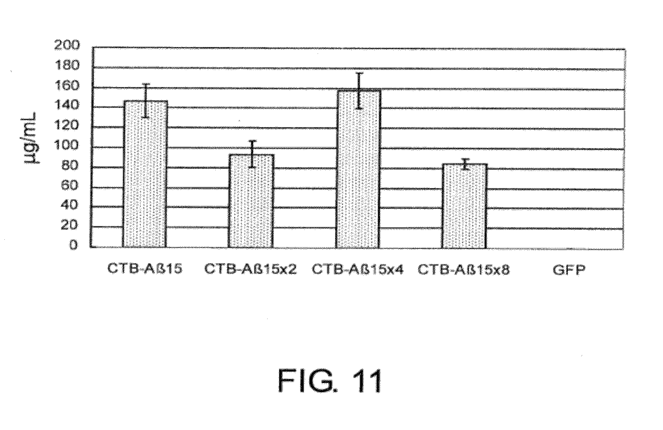

The result showed that the antibody titer was significantly elevated in all administration groups as compared to the control group. In particular, the titer of anti-Aβ antibody was higher in the CTB-Aβ15 and CTB-Aβ15×4 administration groups as compared to the CTB-Aβ15×8 administration group (FIG. 11).

### Example 11

### Assessment of the Variously Constructed SeV Vectors for Booster Effects on the Induction of Anti-Aβ Antibody in Normal Mice

(1) Normal Mice (Intramuscular Administration): Booster Immunization with Purified CTB-Aβ42 Protein

An SeV vector carrying CTB-Aβ42 gene was administered at a titer of 5×10⁷ CIU/head intramuscularly (right hind leg) to C57BL/6N mice. After 14 and 28 days, the CTB-Aβ42 protein produced in E. coli was intramuscularly (right hind leg) administered at 20 μg/PBS/head, 100 μg/PBS/head, or 100 μg/IFA (Freund's incomplete adjuvant)/head to assess the antibody titer. Every 14 days after the treatment, blood was collected from the mice to determine the plasma level of anti-Aβ antibody.

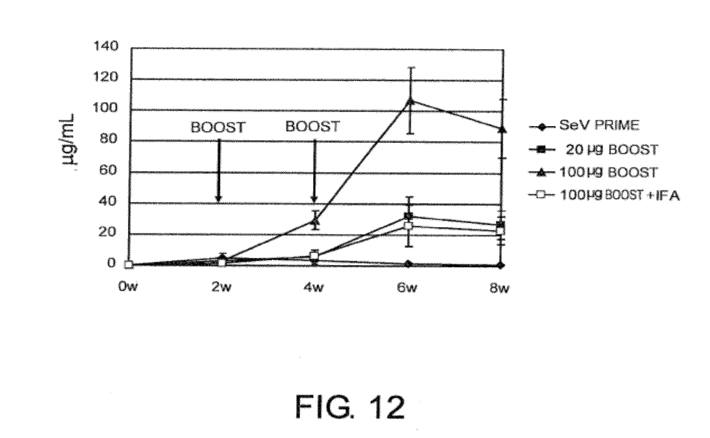

The result showed that the anti-Aβ antibody level was significantly increased in the group immunized with the CTB-Aβ42 gene and booster immunized with the CTB-Aβ42 protein (FIG. 12). After two rounds of booster immunization, the titer of anti-Aβ antibody was 32 μg/ml in the 20-μg booster group, 107 μg/ml in the 100-μg booster group, and 25.9 μg/ml in the [100 μg+IFA] booster group.

(2) Normal Mice (Intramuscular Administration): Booster Immunization with SeV Vector [1]

SeV vectors carrying the CTB-Aβ42 or CTB-Aβ15×8 gene were administered at a titer of 5×10⁷ CIU/head intramuscularly (right hind leg) to C57BL/6N mice, and after 56 days the same SeV vectors were administered intramuscularly (right hind leg) at the same titer to assess the antibody titer.

14 and 28 days after the treatment, blood was collected from the mice to determine the plasma level of anti-Aβ antibody.

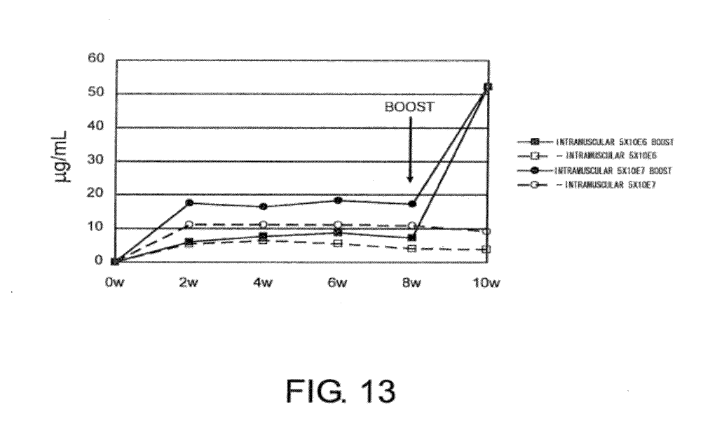

The result showed that the titer of anti-Aβ antibody was significantly elevated in the CTB-Aβ15×8 gene booster group (FIG. 13). Meanwhile, in the CTB-Aβ42 gene booster group, the increase in the anti-Aβ antibody titer was smaller than that of the CTB-Aβ15×8 gene booster group.

(3) Normal Mice (Intramuscular Administration): Booster Immunization with SeV Vectors [2]

SeV vectors carrying the CTB-Aβ15×8 or CTB-Aβ42 gene were administered at a titer of 5×10⁶ or 5×10⁷ CIU/head intramuscularly (right hind leg) to C57BL/6N mice, and after 56 days the same SeV vectors were administered intramuscularly (right hind leg) at the same titer to assess the antibody titer.

14 and 28 days after the treatment, blood was collected from the mice to determine the plasma level of anti-Aβ antibody.

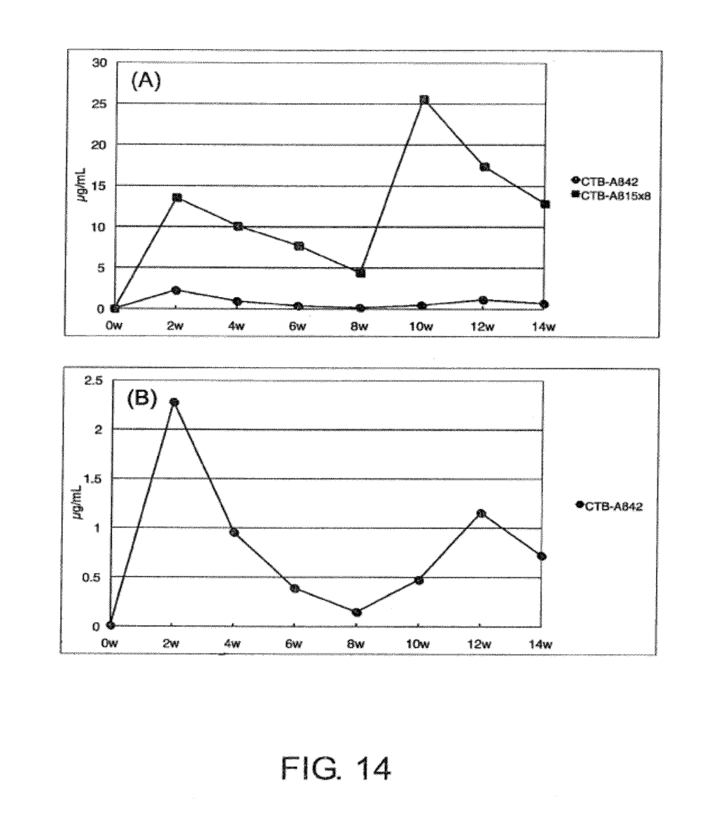

The result showed that clearly both vectors produced the booster effect. In particular, the increase in the anti-Aβ antibody titer was more significant in the CTB-Aβ15×8 gene booster group (FIG. 14).

(4) Normal Mice (Intranasal Administration): Booster Immunization with SeV Vector

An SeV vector carrying the CTB-Aβ15×8 gene was administered at a titer of 5×10⁶ or 5×10⁷ CIU/head intranasally to C57BL/6N mice, and after 56 days the same SeV vector was administered intranasally at the same titer to assess the antibody titer.

14 and 28 days after the treatment, blood was collected from the mice to determine the plasma level of anti-Aβ antibody.

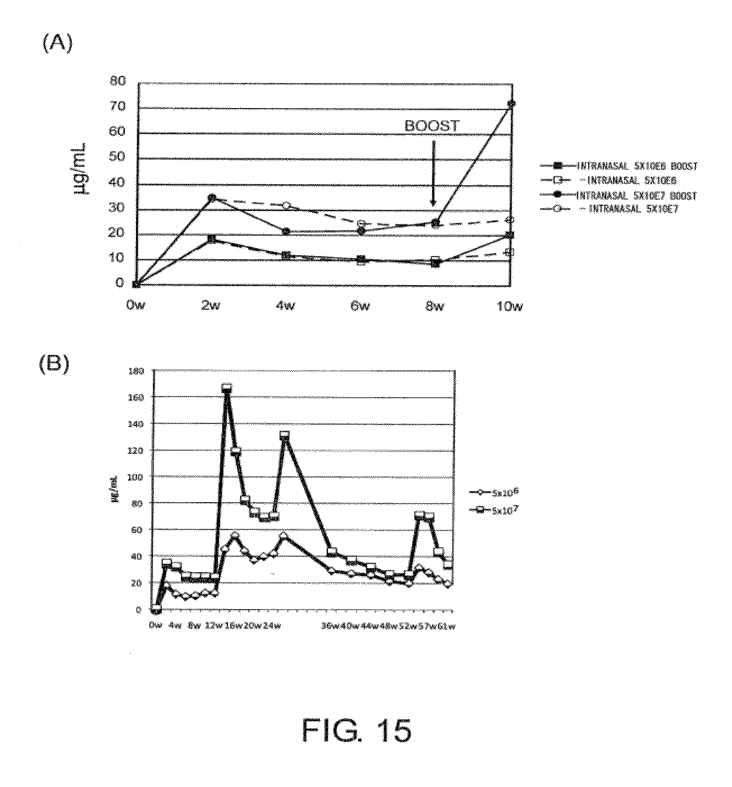

The result showed that the titer of anti-Aβ antibody was significantly elevated in the CTB-Aβ15×8 gene booster group at the rate of 3/3 (FIG. 15A).

(5) Normal Mice (Intranasal Administration): Multiple Rounds of Booster Immunization with SeV Vector and Long-Term Monitoring of Antibody Titer (for One Year)

An SeV vector carrying the CTB-Aβ15×8 gene was administered at a titer of 5×10⁶ or 5×10⁷ CIU/head intranasally to C57BL/6N mice, and 84, 168, and 371 days later the same SeV vector was administered intranasally at the same titer to assess the antibody titer.

14 and 28 days after the treatment, blood was collected from the mice to determine the plasma level of anti-Aβ antibody.

The result showed that the titer of anti-Aβ antibody was significantly elevated in the CTB-Aβ15×8 gene booster group at the rate of 3/3. The antibody titer was maintained at 20 μg/ml or higher one year after the start of the test. Furthermore, the titer of anti-Aβ antibody was significantly elevated by booster immunization carried out one year later (FIG. 15B).

### Example 12

### Assessment of the Constructed Various SeV Vectors for the Effectiveness in APP Model Mice: Intramuscular Administration

### (1) Titer of Anti-Aβ Antibody

SeV18+CTB-Aβ18×5/ΔF (also referred to as CTB-Aβ15×8) or SeV18+CTB-Aβ42/ΔF (also referred to as CTB-Aβ42), or an SeV vector carrying the GFP gene (SeV18+GFP/ΔF; hereinafter also referred to as “GFP”) as control was administered at 5×10⁷ CIU/head intramuscularly (right hind leg) to APP transgenic mice (Tg2576) (13 months old), which served as Alzheimer's disease model mice. After 14 and 28 days, the CTB-Aβ42 protein produced in E. coli was administered intramuscularly (right hind leg) to half of the mice in the CTB-Aβ42 gene administration group. 14, 28, 42, and 56 days after SeV vector administration, assay was carried out to determine the plasma titer of anti-Aβ antibody.

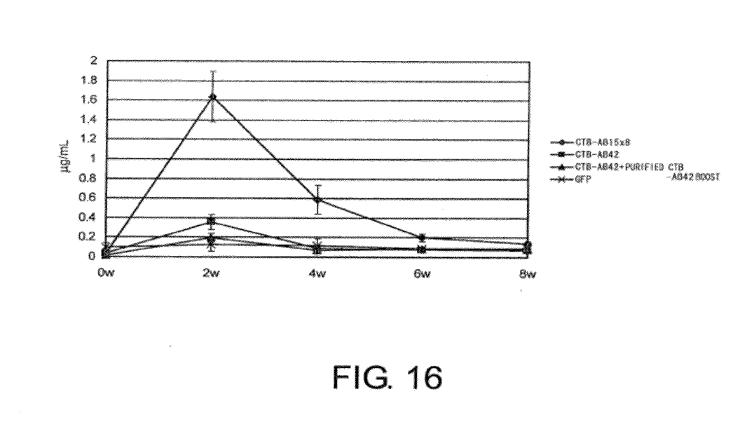

The result showed that the titer of anti-Aβ antibody was significantly elevated in the CTB-Aβ15×8 gene administration group. Meanwhile, in the CTB-Aβ42 gene administration group, the titer of anti-Aβ antibody was only negligibly elevated in half of the mice. The increase in the titer of anti-Aβ antibody was small relative to the CTB-Ali 15×8 gene administration group. The booster immunization did not increase the titer of anti-Aβ antibody in the CTB-Aβ42 protein booster group (FIG. 16).

### (2) Intracerebral Aβ Level: ELISA

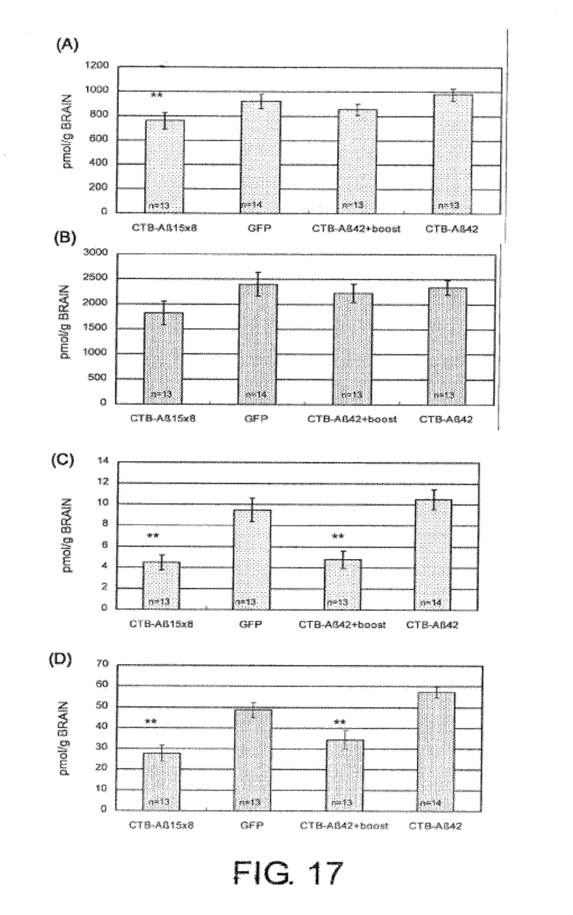

Brain tissues were harvested from the above-described APP mice 56 days after the start of SeV vector administration, and the level of Aβ in the tissue of the left brain hemisphere was determined by ELISA. The brain tissues were homogenized in TBS by sonication. After one hour of centrifugation at 35,000 g, the supernatants were collected as soluble Aβ fractions. Meanwhile, the precipitates were homogenized in 10% formic acid by sonication, and then neutralized with 1 M Tris. The resulting samples were saved as insoluble Aβ fractions. The intracerebral Aβ level was determined using the Aβ42 ELISA kit and Aβ40 ELISA kit (both from Wako Pure Chemical Industries). The result showed that in the CTB-Aβ15×8 gene administration group the Aβ level in the insoluble fraction was reduced to about 80% as compared to the GFP gene administration group. The Aβ level was not reduced in the CTB-Aβ42 gene administration group. Meanwhile, the Aβ level was only slightly reduced in the CTB-Aβ42 protein booster group. In the CTB-Aβ15×8 gene administration group, the Aβ level in the soluble fraction was reduced to about 50% as compared to the GFP gene administration group. The Aβ level was not reduced in the CTB-Aβ42 gene administration group. In the CTB-Aβ42 protein booster group, the Aβ level was reduced to 60% to 70% (FIG. 17).

### (3) Effect of SeV18+CTB-Aβ15×8/ΔF in Eliminating Senile Plaques

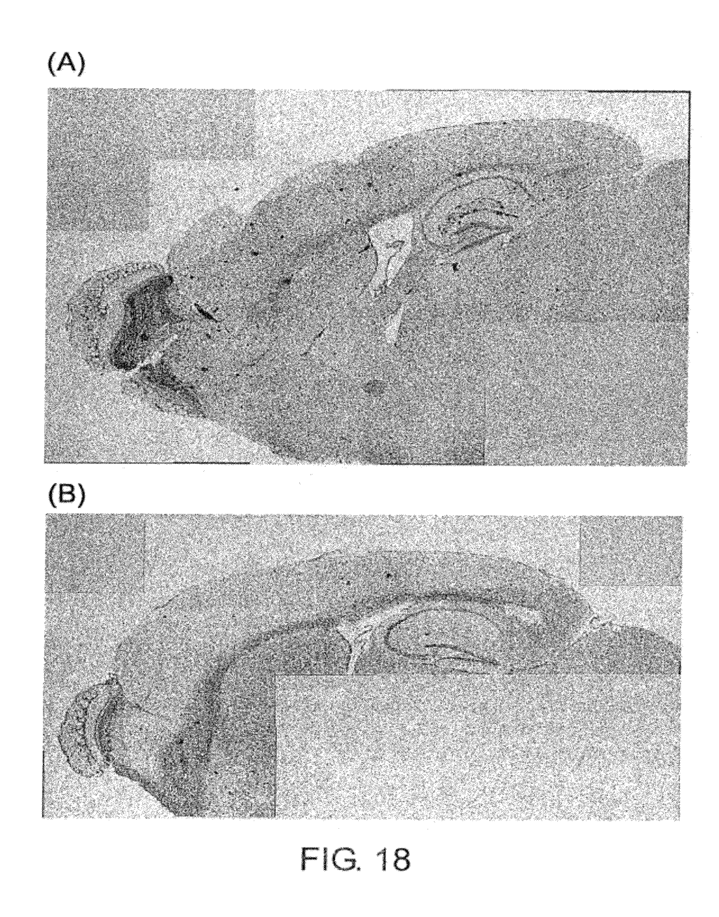

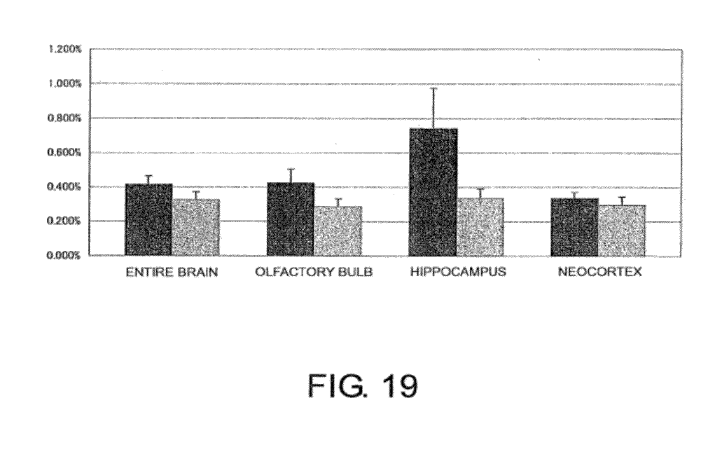

Sendai virus vectors were intramuscularly administered to mice. The mice in each group were dissected eight weeks after administration (15 months old). For histopathological testing, the right brain hemispheres were fixed by immersion in a 10% neutral buffered formalin solution, and after paraffin embedding, longitudinal sections were prepared from the brain tissue located about 2 mm distant from fissura mediana of the brain. The tissue sections were treated with 70% formic acid to detect Aβ protein and senile plaques. The endogenous peroxidase activity was inactivated by 5% H₂O₂. After incubation with an anti-Aβ antibody (antibody 6E10; 1000-fold dilution), a peroxidase-labeled secondary antibody was added for color development with DAB. Then, images were obtained using a 3CCD camera attached to a microscope. For each sample, 20 to 30 image files were combined (FIG. 18). Using image analysis software NIH image, the area of Aβ accumulation in each region of olfactory bulb, cerebral neocortex, and hippocampus was measured under the same conditions for all samples to calculate the ratio of Aβ-accumulated area in each of the regions tested. In addition, the numbers of senile plaques used in the measurement were compared. As seen in FIG. 19, the result showed that the percent area of senile plaques tended to decrease, especially in the hippocampus.

### (4) Safety Assessment of SeV18+CTB-A015×8/ΔF Administration

In the same manner as described in (3), samples of HE- and anti-Iba-1 antibody (microglia)-stained paraffin sections derived from the treatment groups and control group eight weeks after administration (15 months old) were obtained, and they were assessed for the infiltration of inflammatory cells and microglial activation in the central nervous system. The result showed that there was no detectable infiltrating inflammatory cell in any region of the brain both in the control and treatment groups. This demonstrates that the vector of the present invention does not induce inflammation in the central nervous system.

Microglial activation was detected around senile plaques in the animals of both groups. However, in the animals of the vector administration group, the number of senile plaques tended to decrease, and in parallel, the proportion of area occupied with microglias also tended to decrease.

### Example 13

### Construction of an SeV Vector cDNA Containing the NP-Aβ Fusion Protein

By the procedure described below, a Sendai virus vector was constructed to encode a fusion protein having Sendai virus NP protein at its N terminus and Aβ peptides (tandemly linked eight copies of Aβ15 (Aβ15×8)) at its C terminus. PCR was carried out using pSeV18+CTB-Aβ15×8/ΔF as a template and primers SeVF6 (SEQ ID NO: 45) and NP/Aβ15-R (SEQ ID NO: 72) to yield the NP fragment. Primers NP/Aβ15-F (SEQ ID NO: 71) and NotI-EIS-R (SEQ ID NO: 70) were used in PCR to yield the Aβ15×8 fragment. The NP/Aβ15-F and NP/Aβ15-R primers were designed to partially overlap with each other. Thus, the two genes were fused into a single fusion gene by PCR using primers SeVF6 and NotI-EIS-R, and a mixture of NP fragment and Aβ15×8 PCR fragment as template. The resulting PCR fragment was subcloned into a cloning plasmid. After the nucleotide sequence was confirmed, the plasmid was cleaved with restriction enzyme NotI. The resulting NotI fragment which contains the fusion gene of NP-Aβ15×8 was inserted into the NotI site of pSeV18+/ΔF to give the cDNA of an SeV vector containing the NP-Aβ fusion protein of interest (pSeV18+(NP-Aβ15×8)/ΔF).

NotI-EIS-R:
(SEQ ID NO: 70)
5′-ACCTGCGGCCGCGAACTTTCACCCTAAGTTTTTC (34mer)
NP/Aβ15-F:
(SEQ ID NO: 71)
5′-GAATCGGCCCCGGCCCCGACGCCGAGTTCAGACAC (35mer)
NP/Aβ15-R:
(SEQ ID NO: 72)
5′-GCGTCGGGGCCGGGGCCGATTCCTCCTATCCCAGC (35mer)

### Example 14

### Effect of Using the CTB-Aβ Protein (Used Alone or in Combination with SeV Vector)

### (1) Induction of Anti-Aβ Antibody in Normal Mice

CTB-Aβ protein was assessed for induction of anti-Aβ antibody in C57BL/6N mice (8w, female). A gene fragment encoding a fusion protein (CTB-Aβ15×4KK) having CTB at the N terminus and a peptide of four copies of Aβ15 tandemly linked via KK (lysine-lysine) linker at the C terminus was inserted into the NotI site of pSeV18+/ΔF to construct SeV18+CTB-Aβ15×4KK/ΔF. Meanwhile, CTB-Aβ15×4KK was synthesized in E. coli. The vector and the fusion protein were used in the experiment. In group A (six heads), SeV18+GFP/ΔF was intramuscularly administered at 5×10⁷ CIU/200 μl/head, and eight weeks later the same vector was administered in the same manner. In group B (six heads), SeV18+CTB-Aβ15×4KK/ΔF was intramuscularly administered at 5×10⁷ CIU/200 μl/head, and eight weeks later the same vector was administered in the same manner. In group C (six heads), SeV18+CTB-Aβ15×4KK/ΔF was intramuscularly administered at 5×10⁷ CIU/200 μl/head, and then CTB-Aβ15×4KK protein was intracutaneously administered at 100 μg/100 μl/head once every two weeks for a total of five times. In group D (six heads), the CTB-Aβ15×4KK protein was intracutaneously administered at 100 μg/100 μl/head, and then CTB-Aβ15×4KK protein was intracutaneously administered at 100 μg/100 μl/head once every two weeks for a total of five times. Blood was collected from the mice before the initial administration (0 W) and at two-week intervals after administration (2 W-4 W-6 W-8 W-10 W-12 W). The titer of anti-Aβantibody was determined using sera of collected blood samples.

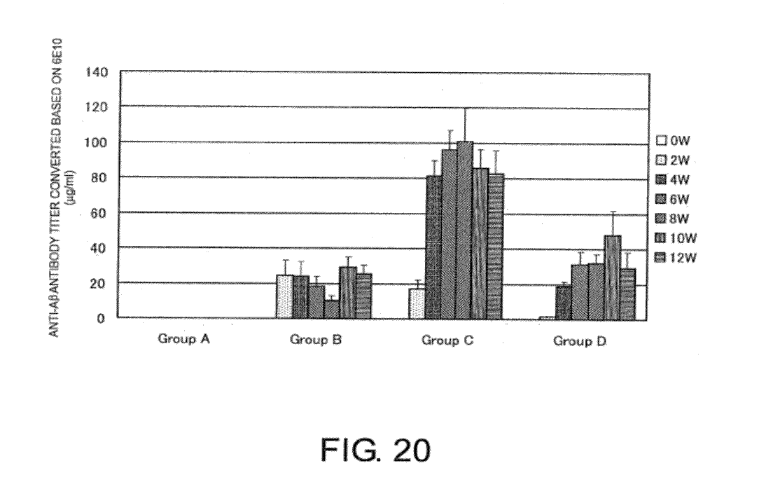

As seen in FIG. 20, the result showed that the combined use of the CTB-Aβ15×4KK protein and SeV (group C) resulted in the induction of anti-Aβ antibody at a very high titer as compared to SeV alone (Group B). Meanwhile, when the protein was used alone (group D), a high titer of anti-Aβ antibody could also be induced.

### (2) Induction of Anti-Aβ Antibody in PDGF-APPV717I Model Mice

Alzheimer's disease model mice PDGF-hAPPV717I (a gift from the Institute of Experimental Animals, Chinese Academy of Medical Sciences; female, 12 months old, seven to nine heads/group) were treated as follows. The mice were untreated in group A, while in group B, SeV18+CTB-Aβ15×4KK/ΔF was intranasally administered at 5×10⁷ CIU/10 μl/head and eight weeks later the same vector was administered in the same manner. In group C, SeV18+CTB-Aβ15×4KK/ΔF was intranasally administered at 5×10⁷ CIU/10 μl/head and then CTB-Aβ15×4KK protein (produced in E. coli) was intranasally administered at 100 μg/15×2 μl/head once every two weeks for a total of seven times. In group D, the CTB-Aβ15×4KK protein was intranasally administered at 100 μg/15×2 μl/head and then CTB-Aβ15×4KK protein was intranasally administered at 100 μg/15×2 μl/head once every two weeks for a total of seven times. Blood was collected from the mice before (0 W) and after (2 W-8 W-12 W-16 W) the initial administration. The titer of anti-Aβ antibody was determined using sera of collected blood samples.

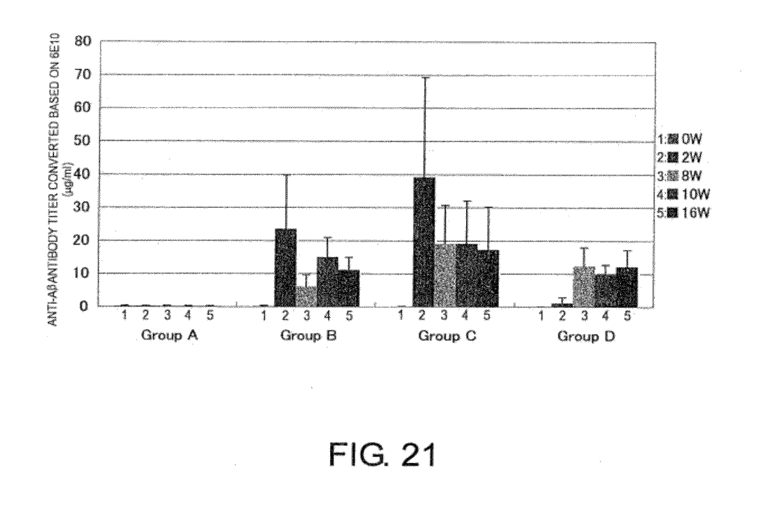

As seen in FIG. 21, the result showed that booster immunization with the CTB-Aβ15×4KK protein (group C) resulted in the induction of anti-Aβ antibody. The protein used alone (group D) could also induce anti-Aβ antibody.

### (3) Assessment Using Tg2576 Mice

Alzheimer's disease model Tg2576 mice (a gift from TACONIC Co., female, 12 months old, 14 to 16 heads/group) were treated as follows. The mice were untreated in group A, while in group B, SeV18+GFP/ΔF was intranasally administered at 5×10⁷ CIU/200 μl/head and 12 weeks later the same vector was administered in the same manner. In group C, SeV18+CTB-Aβ15×4KK/ΔF was intranasally administered at 5×10⁷ CIU/10 μl/head, and then the CTB-Aβ15×4KK protein was intracutaneously administered at 100 μg/100 μl/head once every week for a total of four times, and then once every two weeks for a total of five times. Blood was collected from the mice before (0 W) and after (4 W-8 W-12 W-16 W) administration. The titer of anti-Aβ antibody was determined using sera of collected blood samples. Ultimately, the mice were dissected, and their left brains were fixed in a 10% formalin fixation solution. The brain sections were immunostained (FSB staining and 6E10 staining). The right brains were frozen and stored until extraction of Aβ, and intracerebral Aβ was quantified by ELISA.

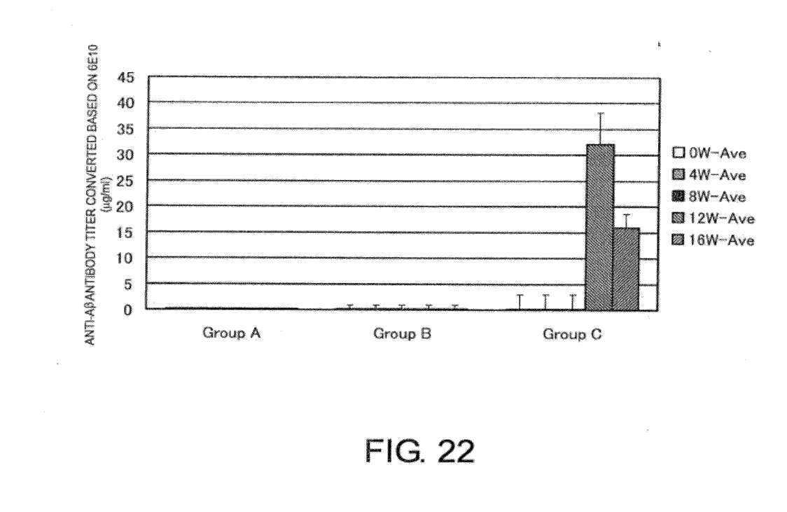

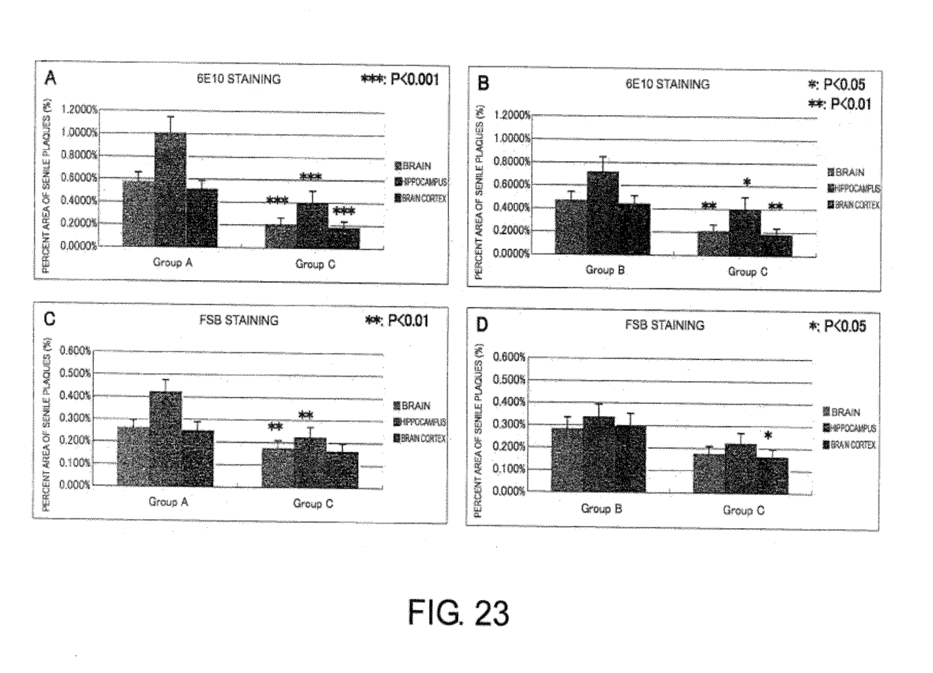

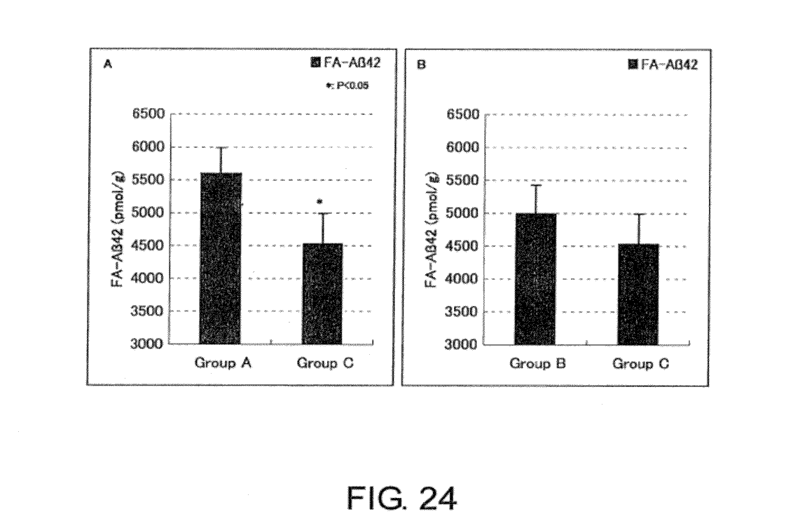

As seen in FIG. 22, the result showed that anti-Aβ antibody was induced at a high titer in group C administered with vaccine (SeV+protein) as compared to untreated group A and control vector administration group B. Furthermore, the result of immunostaining shown in FIG. 23 demonstrated that the area of senile plaques was significantly reduced in group C administered with vaccine as compared to the untreated group (group A) and control vector administration group (group B). Finally, the result of ELISA quantitation of intracerebral Aβshown in FIG. 24 revealed that the insoluble Aβ42 fraction was significantly reduced in group C administered with vaccine as compared to the untreated group.

### Example 15

### Induction of Anti-Aβ Antibody by Various Vectors

PDGF-hAPPV717I model mice (a gift from the Institute of Experimental Animals, Chinese Academy of Medical Sciences; male, 12 months old, 8 or 9 heads/group) were treated as follows. In group A, an adeno-associated virus (AAV) vector that expressed GFP, AAV-GFP, was intramuscularly administered at 5×10¹⁰ particles/200 μl/head, and eight weeks later the same vector was administered in the same manner. In group B, an AAV vector that expressed the CTB-Aβ fusion protein, AAV-CTBAβ42, was intramuscularly administered at 5×10¹⁰ particles/200 μl/head, and eight weeks later the same vector was administered in the same manner. In group C, SeV18+GFP/ΔF was intramuscularly administered at 5×10⁷ CIU/200 μl/head, and eight weeks later the same vector was administered in the same manner. In group D, SeV18+(CTB-Aβ42)/ΔF was intramuscularly administered at 5×10⁷ CIU/200 μl/head, and eight weeks later the same vector was administered in the same manner. In group E, SeV18+CTB-Aβ15×4KK/ΔF was intramuscularly administered at 5×10⁷ CIU/200 μl/head, and eight weeks later the same vector was administered in the same manner. In group F, SeV18+CTB-Aβ15×4KK/ΔF was intranasally administered at 5×10⁷ CIU/10 μl/head, and eight weeks later the same vector was administered in the same manner. Blood was collected from the mice before (0 W) and after (2 W-8 W-12 W-16 W) administration. The titer of anti-Aβ antibody was determined using sera of collected blood samples.

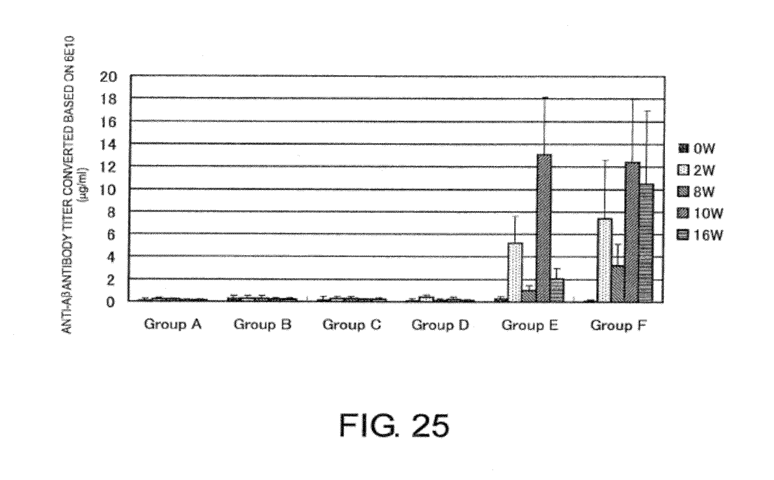

As seen in FIG. 25, the result showed that the SeV vector carrying CTB-A042 induced a slightly higher titer of anti-Aβ antibody as compared to the AAV vector carrying CTB-Aβ42, while the SeV vector (groups E and F) carrying CTB-Aβ15×4KK could induce a remarkably higher titer of anti-Aβ antibody than the two described above (groups B and D).

### Example 16

### Induction of Anti-Aβ Antibody Using a Non-Infectious Viral Vector (VLP)

Non-infectious viral vector (VLP) was assessed for induction of anti-Aβ antibody using C57BL/6N mice (8w, female). In group A (six heads), SeV18+GFP/ΔF was intramuscularly administered at 5×10⁷ CIU/200 μl/head, and then the same vector was administered once every week for a total of four times, and then once after two weeks in the same manner. In group B (six heads), SeV18+(NP-Aβ15×8)/ΔF-VLP, which is a non-infectious particle, was intramuscularly administered at 150 μg/200 μl/head, and then the same vector was administered once every week for a total of four times, and then once after two weeks in the same manner. Blood was collected from the mice before (0 W) and after (2 W-4 W-6 W-8 W) the initial administration. The titer of anti-Aβ antibody was determined using sera of collected blood samples.

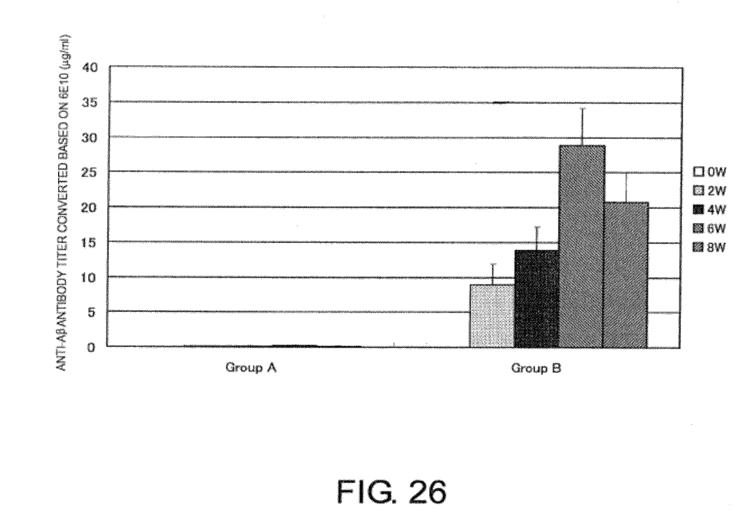

As seen in FIG. 26, the result showed that anti-Aβ antibody could be induced by administering VLP (group B).

### INDUSTRIAL APPLICABILITY

The present invention enables a more effective induction of anti-Aβ antibody. Vaccine therapy for Alzheimer's disease using the present invention is expected not only to help patients with the Alzheimer-type dementia which has no effective therapeutic method, but also to achieve many social contributions such as reduction of medical costs as well as significant improvement of the quality of life and problems of nursing care in elderly persons. The burdens on patients and their families as well as social burdens can be expected to be considerably reduced when radical therapy is provided at early stages by using the highly effective vaccine therapy of the present invention in combination with early diagnosis.

1. An RNA viral vector encoding a fusion protein between an AB5 toxin B subunit and an amyloid β antigen peptide.

2. The vector of claim 1, wherein the AB5 toxin B subunit is a cholera toxin B (CTB).

3. The vector of claim 1, wherein the amyloid β antigen peptide comprises one or more copies of Aβ1-15 or a fragment thereof.

4. The vector of claim 3, wherein the amyloid β antigen peptide has a structure in which one to eight copies of Aβ1-15 or fragments thereof are linked together.

5. The vector of claim 4, wherein the amyloid β antigen peptide has a structure in which four to eight copies of Aβ1-15 are linked together.

6. The vector of claim 1, wherein the RNA viral vector is a minus-strand RNA viral vector.

7. The vector of claim 6, wherein the minus-strand RNA viral vector is a paramyxovirus vector.

8. The vector of claim 7, wherein the paramyxovirus vector is a Sendai virus vector.

9. A composition comprising the vector of claim 1 and a pharmaceutically acceptable carrier.

10. The composition of claim 9 for inducing an anti-Aβ antibody.

11. The composition of claim 9 for preventing or treating Alzheimer's disease.

12. A pharmaceutical agent for preventing or treating Alzheimer's disease, which comprises the vector of claim 1.

13.-16. (canceled)

17. A method for inducing an anti-Aβ antibody, which comprises the step of administering the vector of claim 1 or a composition comprising the vector and a pharmaceutically acceptable carrier.

18. A method for preventing or treating Alzheimer's disease, which comprises the step of administering the vector of claim 1 or a composition comprising the vector and a pharmaceutically acceptable carrier.

19. The method of claim 17, which additionally comprises the step of administering a protein comprising an amyloid β antigen or a vector encoding the protein for booster immunization.

20. The method of claim 19, wherein the protein comprising an amyloid β antigen is a fusion protein between an AB5 toxin B subunit and an amyloid β antigen peptide.

21. A method for increasing the titer of an antibody against an antigen protein, which comprises the step of administering an RNA viral vector encoding the antigen two or more times.

22. A composition for increasing the titer of an antibody against an antigen protein by a method that comprises the step of administering two or more times a vector that comprises an RNA viral vector encoding the antigen and a pharmaceutically acceptable carrier.

23. (canceled)

24. A virus-like particle comprising the vector of claim 1.

25. The method of claim 18, which additionally comprises the step of administering a protein comprising an amyloid β antigen or a vector encoding the protein for booster immunization.

26. The method of claim 25, wherein the protein comprising an amyloid β antigen is a fusion protein between an AB5 toxin B subunit and an amyloid β antigen peptide.


SEQUENCE LISTING (73 sequences)

SEQ ID NO: 1 - protein, 42 aa, Homo sapiens [sequence omitted]

SEQ ID NO: 2 - DNA, 530 bp, Artificial
  an artificially synthesized sequence
gcggccgccaaggttcaatgagcgtgcccacccaggtgctgggcctgctgctgctgtggctgaccgacgccagatgcgacgccgagttcagacacgacagcggctacgaggtgcaccaccagaagctggtgttcttcgccgaggacgtgggcagcaacaagggcgccatcatcggcctgatggtgggcggcgtggtgatcgcctaagaattcacgcgtggtacctctagagtcgacccgggcccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatccaccgtagtaagaaaaacttagggtgaaagttcgcggccgc

SEQ ID NO: 3 - protein, 62 aa, Artificial
  Synthetic Construct
MSVPTQVLGLLLLWLTDARCDAEFRHDSGYEVHHQKLVFFAEDVGSNKGAIIGLMVGGVVIA

SEQ ID NO: 4 - DNA, 45 bp, Artificial
  an artificially synthesized sequence
gcgaattcgccaccatgagcgtgcccacccaggtgctgggcctgc

SEQ ID NO: 5 - DNA, 45 bp, Artificial
  an artificially synthesized sequence
gctacgaggtgcaccaccagaagctggtgttcttcgccgaggacg

SEQ ID NO: 6 - DNA, 45 bp, Artificial
  an artificially synthesized sequence
cgtcgcatctggcgtcggtcagccacagcagcagcaggcccagca

SEQ ID NO: 7 - DNA, 45 bp, Artificial
  an artificially synthesized sequence
gcacctcgtagccgctgtcgtgtctgaactcggcgtcgcatctgg

SEQ ID NO: 8 - DNA, 45 bp, Artificial
  an artificially synthesized sequence
tcaggccgatgatggcgcccttgttgctgcccacgtcctcggcga

SEQ ID NO: 9 - DNA, 45 bp, Artificial
  an artificially synthesized sequence
agaattcttaggcgatcaccacgccgcccaccatcaggccgatga

SEQ ID NO: 10 - DNA, 25 bp, Artificial
  an artificially synthesized sequence
ggcgaattcgccaccatgagcgtgc

SEQ ID NO: 11 - DNA, 25 bp, Artificial
  an artificially synthesized sequence
taagaattcttaggcgatcaccacg

SEQ ID NO: 12 - DNA, 43 bp, Artificial
  an artificially synthesized sequence
atctgcggccgccaaggttcaatgagcgtgcccacccaggtgc

SEQ ID NO: 13 - DNA, 60 bp, Artificial
  an artificially synthesized sequence
acctgcggccgcgatgaactttcaccctaagtttttcttactacggtggatccttatcga

SEQ ID NO: 14 - protein, 124 aa, Vibrio cholerae
MIKLKFGVFFTVLLSSAYAHGTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMAN

SEQ ID NO: 15 - DNA, 800 bp, Artificial
  an artificially synthesized sequence
gcggccgctggctagccaccatgatcaagctgaagttcggcgtgttcttcaccgtgctgctgagcagcgcctacgcccacggcaccccccagaacatcaccgacctgtgcgccgagtaccacaacacccagatccacaccctgaacgacaagatcttcagctacaccgagagcctggccggcaagagagagatggccatcatcaccttcaagaacggcgccaccttccaggtggaggtgcccggctcccagcacatcgacagccagaagaaggccatcgagagaatgaaggacaccctgcggatcgcctacctgaccgaggccaaggtggagaagctgtgcgtgtggaacaacaagaccccccacgccatcgccgccatcagcatggccaacggccccggccccgacgccgagttcagacacgacagcggctacgaggtgcaccaccagaagctggtgttcttcgccgaggacgtgggcagcaacaagggcgccatcatcggcctgatggtgggcggcgtggtgatcgcctaacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtagatccgtagtaagaaaaacttagggtgaaagttcatcgcggccgc

SEQ ID NO: 16 - protein, 170 aa, Artificial
  Synthetic Construct
MIKLKFGVFFTVLLSSAYAHGTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANGPGPDAEFRHDSGYEVHHQKLVFFAEDVGSNKGAIIGLMVGGVVIA

SEQ ID NO: 17 - DNA, 84 bp, Artificial
  an artificially synthesized sequence
cagcggccgctggctagccaccatgatcaagctgaagttcggcgtgttcttcaccgtgctgctgagcagcgcctacgcccacgg

SEQ ID NO: 18 - DNA, 84 bp, Artificial
  an artificially synthesized sequence
acccagatccacaccctgaacgacaagatcttcagctacaccgagagcctggccggcaagagagagatggccatcatcaccttc

SEQ ID NO: 19 - DNA, 85 bp, Artificial
  an artificially synthesized sequence
cacatcgacagccagaagaaggccatcgagagaatgaaggacaccctgcggatcgcctacctgaccgaggccaaggtggagaagc

SEQ ID NO: 20 - DNA, 86 bp, Artificial
  an artificially synthesized sequence
tcagcatggccaacggccccggccccgacgccgagttcagacacgacagcggctacgaggtgcaccaccagaagctggtgttcttc

SEQ ID NO: 21 - DNA, 85 bp, Artificial
  an artificially synthesized sequence
ttcagggtgtggatctgggtgttgtggtactcggcgcacaggtcggtgatgttctggggggtgccgtgggcgtaggcgctgctca

SEQ ID NO: 22 - DNA, 84 bp, Artificial
  an artificially synthesized sequence
cttcttctggctgtcgatgtgctgggagccgggcacctccacctggaaggtggcgccgttcttgaaggtgatgatggccatctc

SEQ ID NO: 23 - DNA, 86 bp, Artificial
  an artificially synthesized sequence
cggggccgttggccatgctgatggcggcgatggcgtggggggtcttgttgttccacacgcacagcttctccaccttggcctcggtc

SEQ ID NO: 24 - DNA, 85 bp, Artificial
  an artificially synthesized sequence
gatcaccacgccgcccaccatcaggccgatgatggcgcccttgttgctgcccacgtcctcggcgaagaacaccagcttctggtgg

SEQ ID NO: 25 - DNA, 30 bp, Artificial
  an artificially synthesized sequence
cagcggccgctggctagccaccatgatcaa

SEQ ID NO: 26 - DNA, 27 bp, Artificial
  an artificially synthesized sequence
tcgcggccgcgatgaactttcacccta

SEQ ID NO: 27 - DNA, 423 bp, Mus musculus
  CDS (1)..(420)
atgggtctcaacccccagctagttgtcatcctgctcttctttctcgaatgtaccaggagccatatccacggatgcgacaaaaatcacttgagagagatcatcggcattttgaacgaggtcacaggagaagggacgccatgcacggagatggatgtgccaaacgtcctcacagcaacgaagaacaccacagagagtgagctcgtctgtagggcttccaaggtgcttcgcatattttatttaaaacatgggaaaactccatgcttgaagaagaactctagtgttctcatggagctgcagagactctttcgggcttttcgatgcctggattcatcgataagctgcaccatgaatgagtccaagtccacatcactgaaagacttcctggaaagcctaaagagcatcatgcaaatggattactcgtag

SEQ ID NO: 28 - protein, 140 aa, Mus musculus
MGLNPQLVVILLFFLECTRSHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS

SEQ ID NO: 29 - DNA, 45 bp, Artificial
  an artificially synthesized sequence
attgcggccgccaaggttcaatgggtctcaacccccagctagttg

SEQ ID NO: 30 - DNA, 44 bp, Artificial
  an artificially synthesized sequence
gtctgaactcggcgtcgctgccgccgcccgagtaatccatttgc

SEQ ID NO: 31 - DNA, 44 bp, Artificial
  an artificially synthesized sequence
gcaaatggattactcgggcggcggcagcgacgccgagttcagac

SEQ ID NO: 32 - DNA, 79 bp, Artificial
  an artificially synthesized sequence
attgcggccgcgatgaactttcaccctaagtttttcttactacggttaggcgatcaccacgccgcccaccatcaggccg

SEQ ID NO: 33 - DNA, 620 bp, Artificial
  an artificially synthesized sequence
gcggccgccaaggttcaatgggtctcaacccccagctagttgtcatcctgctcttctttctcgaatgtaccaggagccatatccacggatgcgacaaaaatcacttgagagagatcatcggcattttgaacgaggtcacaggagaagggacgccatgcacggagatggatgtgccaaacgtcctcacagcaacgaagaacaccacagagagtgagctcgtctgtagggcttccaaggtgcttcgcatattttatttaaaacatgggaaaactccatgcttgaagaagaactctagtgttctcatggagctgcagagactctttcgggcttttcgatgcctggattcatcgataagctgcaccatgaatgagtccaagtccacatcactgaaagacttcctggaaagcctaaagagcatcatgcaaatggattactcgggcggcggcagcgacgccgagttcagacacgacagcggctacgaggtgcaccaccagaagctggtgttcttcgccgaggacgtgggcagcaacaagggcgccatcatcggcctgatggtgggcggcgtggtgatcgcctaaccgtagtaagaaaaacttagggtgaaagttcatcgcggccgc

SEQ ID NO: 34 - protein, 186 aa, Artificial
  Synthetic Construct
MGLNPQLVVILLFFLECTRSHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYSGGGSDAEFRHDSGYEVHHQKLVFFAEDVGSNKGAIIGLMVGGVVIA

SEQ ID NO: 35 - DNA, 99 bp, Artificial
  an artificially synthesized sequence
gccagatgcgccgaggaggccttcgacctgtggaacgagtgcgccaaggcctgcgtgctggacctgaaggacggcgtgagaagcagcagaatgagcgtg

SEQ ID NO: 36 - DNA, 96 bp, Artificial
  an artificially synthesized sequence
gcgtcgttgccgccctccagcaccatgctgtagtgcagcacgccctggccgttggtgtcggcgatggcggggtccacgctcattctgctgcttctc

SEQ ID NO: 37 - DNA, 99 bp, Artificial
  an artificially synthesized sequence
ctggagggcggcaacgacgccctgaagctggccatcgacaacgccctgagcatcaccagcgacggcctgaccatcagactggagggcggcgtggagccc

SEQ ID NO: 38 - DNA, 97 bp, Artificial
  an artificially synthesized sequence
ccgatgggcaccagccagttcaggctccagctgcctctggcctgtctggtgtagctgtatctcacgggcttgttgggctccacgccgccctccagtc

SEQ ID NO: 39 - DNA, 96 bp, Artificial
  an artificially synthesized sequence
gaactggctggtgcccatcggccacgagaagcccagcaacatcaaggtgttcatccacgagctgaacgccggcaaccagctgagccacatgagccc

SEQ ID NO: 40 - DNA, 98 bp, Artificial
  an artificially synthesized sequence
cgtgggctctcacgaagaaggtggcgtctctggccagcttggccagcagctcgtcgcccatctcgatggtgtagatggggctcatgtggctcagctgg

SEQ ID NO: 41 - DNA, 99 bp, Artificial
  an artificially synthesized sequence
caccttcttcgtgagagcccacgagagcaacgagatgcagcccaccctggccatcagccacgccggcgtgagcgtggtgatggcccaggcccagcccag

SEQ ID NO: 42 - DNA, 99 bp, Artificial
  an artificially synthesized sequence
gttgtacacgccgtccagggggtccagcaggcacagcaccttgccgctggcccactcgctccatctcttctctcttctgggctgggcctgggccatcac

SEQ ID NO: 43 - DNA, 96 bp, Artificial
  an artificially synthesized sequence
gaccccctggacggcgtgtacaactacctggcccagcagagatgcaacctggacgacacctgggagggcaagatctacagagtgctggccggcaac

SEQ ID NO: 44 - DNA, 98 bp, Artificial
  an artificially synthesized sequence
cgccgccgccgccctcggggaagtgcagtctgtggctgatcacggtgggcttgatgtccaggtcgtgcttggcggggttgccggccagcactctgtag

SEQ ID NO: 45 - DNA, 22 bp, Artificial
  an artificially synthesized sequence
acaagagaaaaaacatgtatgg

SEQ ID NO: 46 - DNA, 35 bp, Artificial
  an artificially synthesized sequence
cgaaggcctcctcggcgcatctggcgtcggtcagc

SEQ ID NO: 47 - DNA, 35 bp, Artificial
  an artificially synthesized sequence
cgagggcggcggcggcgacgccgagttcagacacg

SEQ ID NO: 48 - DNA, 30 bp, Artificial
  an artificially synthesized sequence
gcatcatcagtcacacttgggcctagtacg

SEQ ID NO: 49 - DNA, 32 bp, Artificial
  an artificially synthesized sequence
gatgcgccgaggaggccttcgacctgtggaac

SEQ ID NO: 50 - DNA, 34 bp, Artificial
  an artificially synthesized sequence
gaactcggcgtcgccgccgccgccctcggggaag

SEQ ID NO: 51 - DNA, 1008 bp, Artificial
  an artificially synthesized sequence
caaggttcaatgagcgtgcccacccaggtgctgggcctgctgctgctgtggctgaccgacgccagatgcgccgaggaggccttcgacctgtggaacgagtgcgccaaggcctgcgtgctggacctgaaggacggcgtgagaagcagcagaatgagcgtggaccccgccatcgccgacaccaacggccagggcgtgctgcactacagcatggtgctggagggcggcaacgacgccctgaagctggccatcgacaacgccctgagcatcaccagcgacggcctgaccatcagactggagggcggcgtggagcccaacaagcccgtgagatacagctacaccagacaggccagaggcagctggagcctgaactggctggtgcccatcggccacgagaagcccagcaacatcaaggtgttcatccacgagctgaacgccggcaaccagctgagccacatgagccccatctacaccatcgagatgggcgacgagctgctggccaagctggccagagacgccaccttcttcgtgagagcccacgagagcaacgagatgcagcccaccctggccatcagccacgccggcgtgagcgtggtgatggcccaggcccagcccagaagagagaagagatggagcgagtgggccagcggcaaggtgctgtgcctgctggaccccctggacggcgtgtacaactacctggcccagcagagatgcaacctggacgacacctgggagggcaagatctacagagtgctggccggcaaccccgccaagcacgacctggacatcaagcccaccgtgatcagccacagactgcacttccccgagggcggcggcggcgacgccgagttcagacacgacagcggctacgaggtgcaccaccagaagctggtgttcttcgccgaggacgtgggcagcaacaagggcgccatcatcggcctgatggtgggcggcgtggtgatcgcctaaccgtagtaagaaaaacttagggtgaaagttcatcgcggccgc

SEQ ID NO: 52 - protein, 318 aa, Artificial
  Synthetic Construct
MSVPTQVLGLLLLWLTDARCAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGGGDAEFRHDSGYEVHHQKLVFFAEDVGSNKGAIIGLMVGGVVIA

SEQ ID NO: 53 - DNA, 34 bp, Artificial
  an artificially synthesized sequence
aacagatatcactggtggtgcacctcgtagccgc

SEQ ID NO: 54 - DNA, 33 bp, Artificial
  an artificially synthesized sequence
atctgatatcagacatgataagatacattgatg

SEQ ID NO: 55 - DNA, 722 bp, Artificial
  an artificially synthesized sequence
gcggccgctggctagccaccatgatcaagctgaagttcggcgtgttcttcaccgtgctgctgagcagcgcctacgcccacggcaccccccagaacatcaccgacctgtgcgccgagtaccacaacacccagatccacaccctgaacgacaagatcttcagctacaccgagagcctggccggcaagagagagatggccatcatcaccttcaagaacggcgccaccttccaggtggaggtgcccggctcccagcacatcgacagccagaagaaggccatcgagagaatgaaggacaccctgcggatcgcctacctgaccgaggccaaggtggagaagctgtgcgtgtggaacaacaagaccccccacgccatcgccgccatcagcatggccaacggccccggccccgacgccgagttcagacacgacagcggctacgaggtgcaccaccagtgatatcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtagatccgtagtaagaaaaacttagggtgaaagttcatcgcggccgc

SEQ ID NO: 56 - protein, 143 aa, Artificial
  Synthetic Construct
MIKLKFGVFFTVLLSSAYAHGTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANGPGPDAEFRHDSGYEVHHQ

SEQ ID NO: 57 - DNA, 29 bp, Artificial
  an artificially synthesized sequence
cggccccggggccgttggccatgctgatg

SEQ ID NO: 58 - DNA, 34 bp, Artificial
  an artificially synthesized sequence
gatgcccgggcagacatgataagatacattgatg

SEQ ID NO: 59 - DNA, 542 bp, Artificial
  an artificially synthesized sequence
gcggccgccaaggttcagaattcgccaccatgagcgtgcccacccaggtgctgggcctgctgctgctgtggctgaccgacgccagatgcgacgccgagttcagacacgacagcggctacgaggtgcaccaccagaagaaggacgccgagttcagacacgacagcggctacgaggtgcaccaccagaagaaggacgccgagttcagacacgacagcggctacgaggtgcaccaccagaagaaggacgccgagttcagacacgacagcggctacgaggtgcaccaccagaagaaggacgccgagttcagacacgacagcggctacgaggtgcaccaccagaagaaggacgccgagttcagacacgacagcggctacgaggtgcaccaccagaagaaggacgccgagttcagacacgacagcggctacgaggtgcaccaccagaagaaggacgccgagttcagacacgacagcggctacgaggtgcaccaccagtaagaattcccgtagtaagaaaaacttagggtgaaagttcatcgcggccgc

SEQ ID NO: 60 - protein, 154 aa, Artificial
  Synthetic Construct
MSVPTQVLGLLLLWLTDARCDAEFRHDSGYEVHHQKKDAEFRHDSGYEVHHQKKDAEFRHDSGYEVHHQKKDAEFRHDSGYEVHHQKKDAEFRHDSGYEVHHQKKDAEFRHDSGYEVHHQKKDAEFRHDSGYEVHHQKKDAEFRHDSGYEVHHQ

SEQ ID NO: 61 - DNA, 29 bp, Artificial
  an artificially synthesized sequence
tggctgaccgatcccgggcccgacgccga

SEQ ID NO: 62 - DNA, 26 bp, Artificial
  an artificially synthesized sequence
ttactacgggatatcttactggtggt

SEQ ID NO: 63 - DNA, 776 bp, Artificial
  an artificially synthesized sequence
gcggccgctggctagccaccatgatcaagctgaagttcggcgtgttcttcaccgtgctgctgagcagcgcctacgcccacggcaccccccagaacatcaccgacctgtgcgccgagtaccacaacacccagatccacaccctgaacgacaagatcttcagctacaccgagagcctggccggcaagagagagatggccatcatcaccttcaagaacggcgccaccttccaggtggaggtgcccggctcccagcacatcgacagccagaagaaggccatcgagagaatgaaggacaccctgcggatcgcctacctgaccgaggccaaggtggagaagctgtgcgtgtggaacaacaagaccccccacgccatcgccgccatcagcatggccaacggccccgggcccgacgccgagttcagacacgacagcggctacgaggtgcaccaccagaagaaggacgccgagttcagacacgacagcggctacgaggtgcaccaccagtaagatgggcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtagatccgtagtaagaaaaacttagggtgaaagttcatcgcggccgc

SEQ ID NO: 64 - protein, 160 aa, Artificial
  Synthetic Construct
MIKLKFGVFFTVLLSSAYAHGTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANGPGPDAEFRHDSGYEVHHQKKDAEFRHDSGYEVHHQ

SEQ ID NO: 65 - DNA, 878 bp, Artificial
  an artificially synthesized sequence
gcggccgctggctagccaccatgatcaagctgaagttcggcgtgttcttcaccgtgctgctgagcagcgcctacgcccacggcaccccccagaacatcaccgacctgtgcgccgagtaccacaacacccagatccacaccctgaacgacaagatcttcagctacaccgagagcctggccggcaagagagagatggccatcatcaccttcaagaacggcgccaccttccaggtggaggtgcccggctcccagcacatcgacagccagaagaaggccatcgagagaatgaaggacaccctgcggatcgcctacctgaccgaggccaaggtggagaagctgtgcgtgtggaacaacaagaccccccacgccatcgccgccatcagcatggccaacggccccgggcccgacgccgagttcagacacgacagcggctacgaggtgcaccaccagaagaaggacgccgagttcagacacgacagcggctacgaggtgcaccaccagaagaaggacgccgagttcagacacgacagcggctacgaggtgcaccaccagaagaaggacgccgagttcagacacgacagcggctacgaggtgcaccaccagtaagatgggcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtagatccgtagtaagaaaaacttagggtgaaagttcatcgcggccgc

SEQ ID NO: 66 - protein, 194 aa, Artificial
  Synthetic Construct
MIKLKFGVFFTVLLSSAYAHGTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANGPGPDAEFRHDSGYEVHHQKKDAEFRHDSGYEVHHQKKDAEFRHDSGYEVHHQKKDAEFRHDSGYEVHHQ

SEQ ID NO: 67 - DNA, 1082 bp, Artificial
  an artificially synthesized sequence
gcggccgctggctagccaccatgatcaagctgaagttcggcgtgttcttcaccgtgctgctgagcagcgcctacgcccacggcaccccccagaacatcaccgacctgtgcgccgagtaccacaacacccagatccacaccctgaacgacaagatcttcagctacaccgagagcctggccggcaagagagagatggccatcatcaccttcaagaacggcgccaccttccaggtggaggtgcccggctcccagcacatcgacagccagaagaaggccatcgagagaatgaaggacaccctgcggatcgcctacctgaccgaggccaaggtggagaagctgtgcgtgtggaacaacaagaccccccacgccatcgccgccatcagcatggccaacggccccgggcccgacgccgagttcagacacgacagcggctacgaggtgcaccaccagaagaaggacgccgagttcagacacgacagcggctacgaggtgcaccaccagaagaaggacgccgagttcagacacgacagcggctacgaggtgcaccaccagaagaaggacgccgagttcagacacgacagcggctacgaggtgcaccaccagaagaaggacgccgagttcagacacgacagcggctacgaggtgcaccaccagaagaaggacgccgagttcagacacgacagcggctacgaggtgcaccaccagaagaaggacgccgagttcagacacgacagcggctacgaggtgcaccaccagaagaaggacgccgagttcagacacgacagcggctacgaggtgcaccaccagtaagatgggcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtagatccgtagtaagaaaaacttagggtgaaagttcatcgcggccgc

SEQ ID NO: 68 - protein, 262 aa, Artificial
  Synthetic Construct
MIKLKFGVFFTVLLSSAYAHGTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANGPGPDAEFRHDSGYEVHHQKKDAEFRHDSGYEVHHQKKDAEFRHDSGYEVHHQKKDAEFRHDSGYEVHHQKKDAEFRHDSGYEVHHQKKDAEFRHDSGYEVHHQKKDAEFRHDSGYEVHHQKKDAEFRHDSGYEVHHQ

SEQ ID NO: 69 - protein, 43 aa, Homo sapiens
DAEFRHDSGYEVHHQKLVFFAEDVGSNKGAIIGLMVGGVVIAT

SEQ ID NO: 70 - DNA, 22 bp, Artificial
  an artificially synthesized sequence
acaagagaaaaaacatgtatgg

SEQ ID NO: 71 - DNA, 34 bp, Artificial
  an artificially synthesized sequence
acctgcggccgcgaactttcaccctaagtttttc

SEQ ID NO: 72 - DNA, 35 bp, Artificial
  an artificially synthesized sequence
gaatcggccccggccccgacgccgagttcagacac

SEQ ID NO: 73 - DNA, 35 bp, Artificial
  an artificially synthesized sequence
gcgtcggggccggggccgattcctcctatcccagc